(19) United States
(12) Patent Application Publication
(10) Pub. No.: US 2026/0298928 A1
(43) Pub. Date: Oct. 1, 2026

(54) METHODS FOR DETECTING ROCAHEPEVIRUS

(71) Applicant: Abbott Laboratories, Abbott Park, IL (US)

(72) Inventors: Michael G. Berg, Abbott Park, IL (US); Abbas Hadji, Abbott Park, IL (US)

(21) Appl. No.: 19/634,748

(22) Filed: Mar. 31, 2026

Related U.S. Application Data
(60) Provisional application No. 63/781,656, filed on Apr. 1, 2025.

(51) Int. Cl. G01N 33/569

(52) U.S. Cl. CPC G01N 33/56983; G01N 2333/08; G01N 2469/20

(57) ABSTRACT

Provided herein are methods, and kits for detecting Rocahepevirus (rHEV) species 7 in human subjects. In certain embodiments, provided herein are Rocahepevirus species 7 specific peptides, and methods for detecting Rocahepevirus species 7 antibodies or antigen-binding fragments thereof in human subjects.

### RELATED APPLICATION INFORMATION

This application claims priority to U.S. Application No. 63/781,656, filed on Apr. 1, 2025, the contents of which are herein incorporated by reference.

### SEQUENCE LISTING STATEMENT

The contents of the electronic sequence listing titled ABBTL_44889_202_SequenceListing.xml (Size: 12,476 bytes; and Date of Creation: Mar. 31, 2026) is herein incorporated by reference in its entirety.

### TECHNICAL FIELD

Provided herein are methods, and kits for detecting Rocahepevirus (rHEV) species 7 in human subjects. In certain embodiments, provided herein are Rocahepevirus species 7 specific peptides, and methods for detecting Rocahepevirus species 7 antibodies or antigen-binding fragments thereof in human subjects.

### BACKGROUND

Viral hepatitis continues to be a major cause of morbidity and mortality globally. Chronic, blood-borne infections due to hepatitis B virus (HBV) and hepatitis C virus (HCV) are collectively responsible for 1.3 million deaths per year (WHO 2024). Hepatitis delta virus (HDV) is a co-infection in about 5% of chronic HBV cases whose prevalence and propensity for exacerbating illness are increasingly being recognized (Lombardo et al., “Hepatitis Delta Virus and Hepatocellular Carcinoma”, Pathogens, 13(5), 2024). Acute, food- or water-borne infections due to hepatitis A virus (HAV) and hepatitis E virus (HEV) are usually self-limiting, but they too represent a significant burden of disease worldwide, with 1.5 million and 20 million cases, respectively, annually (Gabrielli et al., “Treatment Options for Hepatitis A and E: A Non-Systemic Review”, Viruses, 15(5), 2023). Non A-E viruses make up ~20% of total hepatitis cases, which in some instances can be attributed to other common viral infections (e.g., herpesvirus, parvovirus), but often the offending agent goes undiagnosed (Brennan et al., “Systematic review: non A-E, seronegative or indeterminate hepatitis; what is this deadly disease?”, Aliment Pharmacol. Ther., 47(8):1079-91, 2018).

Most strains of HEV infecting humans belong to the species Paslahepevirus balayani (once referred to as Orthohepevirus A; HEV-A), which consists of 8 genotypes (Purdy et al., “ICTV Virus Taxonomy Profile: Hepeviridae 2022”, J. Gen. Virol., 103(9), 2022). Genotypes 1-2 are endemic to Africa and Asia and result from fecal-oral transmission due to poor sanitation and hygiene, whereas genotypes 3, 4 & 7 are more widespread geographically and result from close contact with, or consuming uncooked meat of, infected animals such as swine. The Hepeviridae family also includes genera restricted to bird (Avihepevirus), rat (Rocahepevirus), and bat (Chirohepevirus) hosts, formerly HEV-B/C/D, respectively (Smith et al., “Consensus proposals for classification of the family Hepeviridae”, J Gen Virol, 95 (Pt 10), 2223-32, 2014). Rocahepevirus (rHEV) was discovered in rats in 2010 in Germany (Johne et al., “Novel hepatitis E virus genotype in Norway rats, Germany”, Emerg Infect Dis, 16 (9), 1452-5, 2010), then later detected throughout Europe (Ryll et al., “Detection of rat hepatitis E virus in wild Norway rats (Rattus norvegicus) and Black rats (Rattus rattus) from 11 European countries”, Vet Microbiol, 208, 58-68, 2017) and more broadly worldwide (see (Benavent et al., “Rocahepevirus ratti as an Emerging Cause of Acute Hepatitis Worldwide”, Microorganisms, 11 (12), 2023) and references therein). Related viruses have also been detected in voles, shrews, foxes, ferrets, and other mammals. (Bai et al., “Characterization of a Novel Rat Hepatitis E Virus Isolated from an Asian Musk Shrew (Suncus murinus)”, Viruses, 12 (7), 2020; Lanszki et al., “Identification of Hepatitis E Virus in the Feces of Red Foxes (Vulpes vulpes)”, Animals (Basel), 10 (10), 2020; Reuter et al., “Review of Hepatitis E Virus in Rats: Evident Risk of Species Orthohepevirus C to Human Zoonotic Infection and Disease”, Viruses, 12 (10), 2020; Ryll et al., “Genomic and spatial variability of a European common vole hepevirus”, Arch Virol, 164 (11), 2671-82. 2019).

Though originally believed to be restricted to rodents and lower mammals, Rocahepevirus was first detected in humans in 2018 in a liver transplant patient from Hong Kong with chronic hepatitis (Sridhar et al., “Rat Hepatitis E Virus as Cause of Persistent Hepatitis after Liver Transplant”, Emerg Infect Dis, 24 (12), 2241-50, 2018). The authors went on to identify seven more cases in Hong Kong (Sridhar et al., “Transmission of Rat Hepatitis E Virus Infection to Humans in Hong Kong: A Clinical and Epidemiological Analysis”, Hepatology, 73 (1), 10-22, 2021), and since then, most human infections (16/21; 76%), and rodent rHEV sequences (586/817; 71%) have been found in Asia (Benavent et al., “Rocahepevirus ratti as an Emerging Cause of Acute Hepatitis Worldwide”, Microorganisms, 11 (12), 2023; Wu et al., “Molecular epidemiology and phylogeny of the emerging zoonotic virus Rocahepevirus: A global genetic analysis, Infect Genet Evol, 118, 105557, 2024). Experiments in human cell culture suggest virus shed in human fecal samples lead to abortive infections, whereas virus derived directly from rat liver homogenate still replicated efficiently in several human liver cell lines (Jirintai et al., “‘Rat hepatitis E virus derived from wild rats (Rattus rattus) propagates efficiently in human hepatoma cell lines’, Virus Res, 185, 92-102”, 2014; Schemmerer et al., “HuH-7-Lunet BLR Cells Propagate Rat Hepatitis E Virus (HEV) in a Cell Culture System Optimized for HEV”, Viruses, 14 (5), 2022; Sridhar et al., “Rat Hepatitis E Virus as Cause of Persistent Hepatitis after Liver Transplant”, Emerg Infect Dis, 24 (12), 2241-50, 2018). Against the backdrop of detection in a variety of sample types (Sridhar et al., “Rat Hepatitis E Virus as Cause of Persistent Hepatitis after Liver Transplant”, Emerg Infect Dis, 24 (12), 2241-50, 2018) in multiple patient cases from different countries (Benavent et al., “Rocahepevirus ratti as an Emerging Cause of Acute Hepatitis Worldwide”, Microorganisms, 11 (12), 2023; Reuter et al., “Review of Hepatitis E Virus in Rats: Evident Risk of Species Orthohepevirus C to Human Zoonotic Infection and Disease”, Viruses, 12 (10), 2020), the consensus is that this is indeed a virus with clear zoonotic potential.

The clinical picture that has emerged is that immunosuppressed individuals (solid organ transplant recipients, cancer patients, those with underlying chronic disease such as diabetes or hypertension) are at greatest risk of infection (Benavent et al., “Rocahepevirus ratti as an Emerging Cause of Acute Hepatitis Worldwide”, Microorganisms, 11 (12), 2023). Nevertheless, immunocompetent people are still susceptible, as evidenced by a previously healthy Canadian individual contracting the virus while working for the United Nations in central Africa (DRC or Gabon) (Andonov et al., “Rat Hepatitis E Virus Linked to Severe Acute Hepatitis in an Immunocompetent Patient”, J Infect Dis, 220 (6), 951-55, 2019) and two previously healthy individuals from Spain reporting acute hepatitis (Rivero-Juarez et al., “‘Orthohepevirus C infection as an emerging cause of acute hepatitis in Spain: First report in Europe, J Hepatol, 77 (2), 326-31, 2022). rHEV infection resembles that from HEV-A in terms of its acute and self-limiting nature, often accompanied by fever, fatigue, nausea, and abdominal pain, but it can also persist chronically or present sub-clinically (Benavent et al., “Rocahepevirus ratti as an Emerging Cause of Acute Hepatitis Worldwide”, Microorganisms, 11 (12), 2023). Unfortunately, one of the seven cases from Hong Kong died of meningoencephalitis (Sridhar et al., ‘Transmission of Rat Hepatitis E Virus Infection to Humans in Hong Kong: A Clinical and Epidemiological Analysis’, Hepatology, 73 (1), 10-22, 2021), and the third case in the Spanish cohort with metastatic oral cancer died of renal and liver failure (Rivero-Juarez et al., ‘Orthohepevirus C infection as an emerging cause of acute hepatitis in Spain: First report in Europe’, J Hepatol, 77 (2), 326-31, 2022), each potentially attributable to rHEV infection. Also like HEV-A, rHEV is presumably transmitted through contaminated food or water, with virus shed in rat urine and feces (Sridhar et al., “‘Rat Hepatitis E Virus as Cause of Persistent Hepatitis after Liver Transplant”, Emerg Infect Dis, 24 (12), 2241-50, 2018). As all human cases have been detected in the last six years (2018-2024), and with estimates that up to 28% of rats in China (Li et al., “High prevalence of rat hepatitis E virus in wild rats in China’, Vet Microbiol, 165 (3-4), 275-80, 2013) and 30% in Spain (A. Juarez, unpublished) carry the virus, this epidemiologic picture will continue to evolve as we learn more about this virus.

Thus, there is need for methods and kits for diagnosing Rocahepevirus, particularly Rocahepevirus ratti, in human subjects.

### SUMMARY

In one embodiment, the present disclosure relates to a method for detecting a presence of or determining an amount of a Rocahepevirus species 7 antibody or an antigen-binding fragment thereof in a subject. In some aspects, the method comprising the steps of:
a) contacting at least one biological sample from the subject, either simultaneously or sequentially, in any order, with
at least four types of capture specific binding partners, wherein each of the four capture specific binding partners specifically binds to a Rocahepevirus species 7 antibody or an antigen-binding fragment thereof in the sample; and further wherein the four types of capture specific binding partners comprises: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof, and at least one type of detection specific binding partner comprising a detectable label,
thereby producing one or more types of first complexes comprising the four capture specific binding partners-anti-Rocahepevirus species 7 antibody-detection specific binding partner; and
b) assessing a signal from the one or more types of first complexes, wherein the amount of detectable signal from the detectable label indicates the presence or amount of at least one type of anti-Rocahepevirus species 7 antibody or an antigen-binding fragment thereof in the sample.

In some aspects in the above method, the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

In some aspects in the above method, the biological sample is whole blood, plasma, or serum.

In yet other aspects in the above method, the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) and an anti-species IgM (e.g., anti-human-IgM IgG) antibody.

In still other aspects in the above method, the each of the four capture specific binding partner are immobilized on a solid support.

In yet further aspects, the above method is performed in from about 5 to about 20 minutes, less than about 20 minutes, and optionally is performed in less than about 5 minutes, less than about 10 minutes or less than about 15 minutes.

In yet further aspects, the above method further comprises use with at least one calibrator reagent, at least one control reagent, or at least one calibrator reagent and at least one control reagent.

In some aspects, the above method is an immunoassay or a clinical chemistry assay.

In still further aspects, the above method is performed using single molecule detection, lateral flow assay, or a point-of-care assay.

In another embodiment, the present disclosure relates to a method for performing the above method. The kit used to perform the above method comprises:
a. at least four types of capture specific binding partners, wherein each of the four capture specific binding partners specifically binds to a Rocahepevirus species 7 antibody or an antigen-binding fragment thereof in a sample; and further wherein the four types of capture specific binding partners comprise: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof; and
b. at least one type of detection specific binding partner comprising at least one detectable label.

In some aspects of the above kit, the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

In yet still further aspects, the above kit further comprises, or is configured to be used with, at least one calibrator reagent, at least one control reagent, or at least one calibrator reagent and at least one control reagent.

In yet still further aspects, the above kit further comprises at least one solid support.

In yet still other aspects of the above kit, the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) antibody and an anti-species IgM (e.g., anti-human-IgM IgG) antibody.

In yet still further aspects of the above method, the kit is adapted for use with an automated or semi-automated system.

### BRIEF DESCRIPTION OF THE DRAWINGS

FIG. 1 shows the genome organization structure of rat hepatitis E virus and localization of the coding sequence of the recombinant rat HEV capsid protein used for the generation of the isolated recombinant antigens of SEQ ID NOS: 1-4 (Full length open reading frame 2) and SEQ ID NOS: 5-8 (p166).

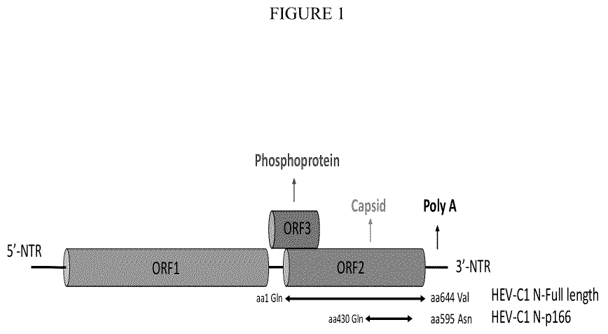

FIG. 2 shows the consensus sequences of 4 Rocahepevirus species 7 open reading frame (2), including two from genotype 1 and two from genotype 2, used to create the isolated recombinant antigens shown in SEQ ID NOS: 1-8 as described herein.

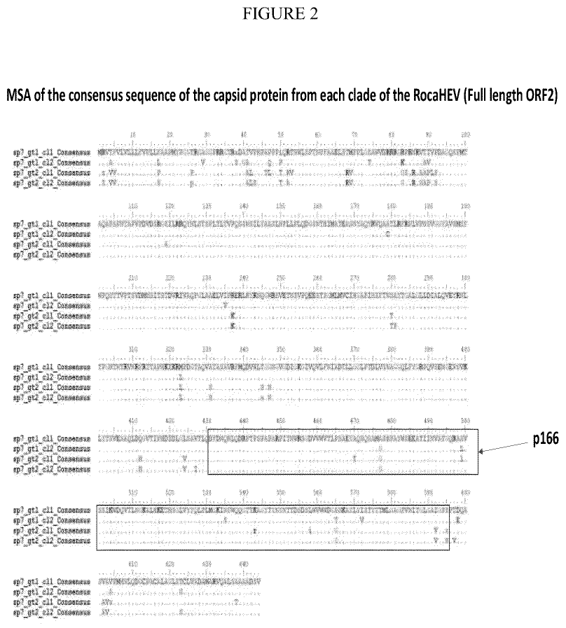

FIG. 3 shows the method of the present disclosure shown as a 2-step indirect IgG assay.

### DETAILED DESCRIPTION

In some aspects, provided herein are provided herein are methods and kits for detecting Rocahepevirus species 7 infection in a subject.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. Antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody” (e.g., an anti-UCH-L1 antibody or a UCH-L1 antibody).

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

The “area under curve” or “AUC” refers to area under a ROC curve. AUC under a ROC curve is a measure of accuracy. An AUC of 1 represents a perfect test, whereas an AUC of 0.5 represents an insignificant test. A preferred AUC may be at least approximately 0.700, at least approximately 0.750, at least approximately 0.800, at least approximately 0.850, at least approximately 0.900, at least approximately 0.910, at least approximately 0.920, at least approximately 0.930, at least approximately 0.940, at least approximately 0.950, at least approximately 0.960, at least approximately 0.970, at least approximately 0.980, at least approximately 0.990, or at least approximately 0.995.

The terms “bead,” “microparticle,” and “particle” used herein interchangeably herein, refer to a substantially spherical solid support. The bead may be a magnetic bead or a magnetic particle. Microparticles that can be used herein can be any type known in the art. For example, the bead or microparticle can be a magnetic bead or magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO·Fe₂O₃). Beads can have a solid core portion that is magnetic and is surrounded by one or more non-magnetic layers. Alternately, the magnetic portion can be a layer around a non-magnetic core. The size of the microparticles used in the methods, compositions and kits described herein can vary. Preferably, the microparticles have a substantially uniform diameter of less than about 0.10 μm, less than about 0.20 μm, less than about 0.30 μm, less than about 0.40 μm, less than about 0.50 μm, less than about 0.60 μm, less than about 0.70 μm, less than about 0.80 μm, less than about 0.9 μm, less than about 1 μm, less than about 2 μm, less than about 3 μm, less than about 4 μm, or less than about 5 μm.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, an IgG molecule, an IgG1 molecule, a monoclonal antibody, a chimeric antibody, a CDR-grafted antibody, a humanized antibody, an affinity matured antibody, and fragments of any such antibodies that retain the ability to bind to an antigen.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds to.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196:901-917 (1987); and Chothia et al., Nature, 342:877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9:133-139 (1995), and MacCallum, J. Mol. Biol., 262 (5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Compiling” refers to a process in which a subject's diagnostic criteria is organized to facilitate diagnosing or aiding in the diagnosis of Rocahepevirus species 7 in a subject. In some aspects, the diagnostic criteria can be compiled electronically. In other aspects, the diagnostic criteria can be complied manually.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay. A “conformational epitope” is an epitope that is comprised of specific juxtaposition of amino acids in an immunologically recognizable structure, such amino acids being present on the same polypeptide in a contiguous or non-contiguous order or present on different polypeptides.

A “conformational epitope” is an epitope that is comprised of specific juxtaposition of amino acids in an immunologically recognizable structure, such amino acids being present on the same polypeptide in a contiguous or non-contiguous order or present on different polypeptides.

“Correlated to” as used herein refers to compared to.

“Determined by an assay” is used herein to refer to the determination of a reference level by any appropriate assay. The determination of a reference level may, in some embodiments, be achieved by an assay of the same type as the assay that is to be applied to the sample from the subject (for example, by an immunoassay, clinical chemistry assay, a single molecule detection assay, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS)). The determination of a reference level may, in some embodiments, be achieved by an assay of the same type and under the same assay conditions as the assay that is to be applied to the sample from the subject. As noted herein, this disclosure provides exemplary reference levels (e.g., calculated by comparing reference levels at different time points). It is well within the ordinary skill of one in the art to adapt the disclosure herein for other assays to obtain assay-specific reference levels for those other assays based on the description provided by this disclosure. It will be understood that a reference level “determined by an assay” and having a recited level of “sensitivity” and/or “specificity” is used herein to refer to a reference level which has been determined to provide a method of the recited sensitivity and/or specificity when said reference level is adopted in the methods of the invention. It is well within the ordinary skill of one in the art to determine the sensitivity and specificity associated with a given reference level in the methods of the invention, for example by repeated statistical analysis of assay data using a plurality of different possible reference levels.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

As used herein, the phrase, “directly detectable,” when used in reference to a detectable label or detectable moiety, means that the detectable label or detectable moiety does not require further reaction or manipulation to be detectable. For example, a fluorescent moiety is directly detectable by fluorescence spectroscopy methods. In contrast, the phrase “indirectly detectable,” when used herein in reference to a detectable label or detectable moiety, means that the detectable label or detectable moiety becomes detectable after further reaction or manipulation. For example, a hapten becomes detectable after reaction with an appropriate antibody attached to a reporter, such as a fluorescent dye.

“Dual-specific antibody” is used herein to refer to a full-length antibody that can bind two different antigens (or epitopes) in each of its two binding arms (a pair of HC/LC) (see PCT publication WO 02/02773). Accordingly, a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig.” Such a DVD-Ig binding protein is thus tetrameric and reminiscent of an IgG molecule but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25:1290-1297 (2007). A preferred example of such DVD-Ig molecules comprises a heavy chain that comprises the structural formula VD1-(X1)n-VD2-C—(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C—(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In a preferred embodiment, a DVD-Ig binding protein not only binds the same target molecules bound by its parental monoclonal antibodies, but also possesses one or more desirable properties of one or more of its parental monoclonal antibodies. Preferably, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

“Dynamic range” as used herein refers to range over which an assay readout is proportional to the amount of target molecule or analyte in the sample being analyzed.

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

A “fragment” of a specified polypeptide refers to portions of an amino acid sequence. Fragments may range in size from 5 amino acid residues to the entire amino acid sequence minus one amino acid residue. In some aspects, the fragment is 5-10 amino acids in length, 5-15 amino acids in length, 5-20 amino acids in length, 5-25 amino acids in length, 5-30 amino acids in length, 5-35 amino acids in length, 5-40 amino acids in length, 5-45 amino acids in length, 5-50 amino acids in length, 5-55 amino acids in length, 5-60 amino acids in length, 5-65 amino acids in length, 5-70 amino acids in length, 5-75 amino acids in length, 5-80 amino acids in length, 5-85 amino acids in length, 5-90 amino acids in length, 5-95 amino acids in length, 5-100 amino acids in length, 5-105 amino acids in length, 5-110 amino acids in length, 5-115 amino acids in length, 5-120 amino acids in length, 5-125 amino acids in length, 5-130 amino acids in length, 5-135 amino acids in length, 5-140 amino acids in length, 5-145 amino acids in length, 5-150 amino acids in length, 5-155 amino acids in length, 5-160 amino acids in length, 5-165 amino acids in length, 5-170 amino acids in length, 5-175 amino acids in length, 5-180 amino acids in length, 5-185 amino acids in length, 5-190 amino acids in length, 5-195 amino acids in length, 5-200 amino acids in length, 5-205 amino acids in length, 5-210 amino acids in length, 5-215 amino acids in length, 5-220 amino acids in length, 5-225 amino acids in length, 5-230 amino acids in length, 5-235 amino acids in length, 5-240 amino acids in length, 5-245 amino acids in length, 5-250 amino acids in length, 5-255 amino acids in length, 5-260 amino acids in length, 5-265 amino acids in length, 5-270 amino acids in length, 5-275 amino acids in length, 5-280 amino acids in length, 5-285 amino acids in length, 5-290 amino acids in length, 5-295 amino acids in length, 5-300 amino acids in length, 5-305 amino acids in length, 5-310 amino acids in length, 5-315 amino acids in length, 5-320 amino acids in length, 5-325 amino acids in length, 5-330 amino acids in length, 5-335 amino acids in length, 5-340 amino acids in length, 5-345 amino acids in length, 5-350 amino acids in length, 5-355 amino acids in length, 5-360 amino acids in length, 5-365 amino acids in length, 5-370 amino acids in length, 5-375 amino acids in length, 5-380 amino acids in length, 5-385 amino acids in length, 5-390 amino acids in length, 5-395 amino acids in length, 5-400 amino acids in length, 5-405 amino acids in length, 5-410 amino acids in length, 5-415 amino acids in length, 5-420 amino acids in length, 5-425 amino acids in length, 5-430 amino acids in length, 5-435 amino acids in length, 5-440 amino acids in length, 5-445 amino acids in length, 5-450 amino acids in length, 5-455 amino acids in length, 5-460 amino acids in length, 5-465 amino acids in length, 5-470 amino acids in length, 5-475 amino acids in length, 5-480 amino acids in length, 5-485 amino acids in length, 5-490 amino acids in length, 5-495 amino acids in length, 5-500 amino acids in length, 5-505 amino acids in length, 5-510 amino acids in length, 5-515 amino acids in length, 5-520 amino acids in length, 5-525 amino acids in length, 5-530 amino acids in length, 5-535 amino acids in length, 5-540 amino acids in length, 5-545 amino acids in length, 5-550 amino acids in length, 5-555 amino acids in length, 5-560 amino acids in length, 5-565 amino acids in length, 5-570 amino acids in length, 5-575 amino acids in length, 5-580 amino acids in length, 5-585 amino acids in length, 5-590 amino acids in length, 5-595 amino acids in length, 5-600 amino acids in length, 5-605 amino acids in length, 5-610 amino acids in length, 5-615 amino acids in length, 5-620 amino acids in length, 5-625 amino acids in length, 5-630 amino acids in length, 5-635 amino acids in length, 5-640 amino acids in length, 5-645 amino acids in length, or 5-650 amino acids in length.

“Fragment antigen-binding fragment”, “antigen-binding fragment”, or “Fab fragment” as used herein refers to a fragment of an antibody that binds to antigens and that contains one antigen-binding site, one complete light chain, and part of one heavy chain. Fab is a monovalent fragment consisting of the VL, VH, CL and CH1 domains. Fab is composed of one constant and one variable domain of each of the heavy and the light chain. The variable domain contains the paratope (the antigen-binding site), comprising a set of complementarity determining regions, at the amino terminal end of the monomer. Each arm of the Y thus binds an epitope on the antigen. Fab fragments can be generated such as has been described in the art, e.g., using the enzyme papain, which can be used to cleave an immunoglobulin monomer into two Fab fragments and an Fc fragment, or can be produced by recombinant means.

“F(ab′)₂ fragment” as used herein refers to antibodies generated by pepsin digestion of whole IgG antibodies to remove most of the Fc region while leaving intact some of the hinge region. F(ab′)₂ fragments have two antigen-binding F (ab) portions linked together by disulfide bonds, and therefore are divalent with a molecular weight of about 110 kDa. Divalent antibody fragments (F(ab′)₂ fragments) are smaller than whole IgG molecules and enable a better penetration into tissue thus facilitating better antigen recognition in immunohistochemistry. The use of F(ab′)₂ fragments also avoid unspecific binding to Fc receptor on live cells or to Protein A/G. F(ab′)₂ fragments can both bind and precipitate antigens.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base (hypertext transfer protocol://vbase.mrc-cpe.cam.ac.uk/) or in the international ImMunoGeneTics® (IMGT®) information system (hypertext transfer protocol://imgt.cines.fr/texts/IMGTrepertoire/LocusGenes/).

“Functional antigen binding site” as used herein may mean a site on a binding protein (e.g., an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“Health care practitioner” refers to any medical professional that has been trained to observe, determine, and document one or more diagnostic criteria of a subject having or suspected of having Rocahepevirus. The health care practitioner may also be trained to adjudicate a Rocahepevirus diagnosis based on the diagnostic criteria, or to use a system that implements a computer-implemented version of the method to adjudicate a Rocahepevirus diagnosis of the subject. Examples of such health care practitioners include a paramedic, a nurse (e.g., a triage nurse), a nurse practitioner, a physician's assistant, a physician, a medical student operating under the supervision of a licensed physician, a specialist, a clinical trial investigator, a principal investigator, etc. Other individuals, such as, for example, a radiologist, a phlebotomist, a laboratory technician may be involved together with a health care practitioner in observing, determining, and/or documenting one or more diagnostic criteria of the subject having or suspected of having Rocahepevirus species 7.

“Humanized antibody” is used herein to describe an antibody that comprises heavy and light chain variable region sequences from a non-human species (e.g., a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

As used herein, the term, “isolated” means that the referenced material, such as, for example, biological material (e.g., DNA, cDNA, RNA, mRNA, a nucleic acid, nucleotide, oligonucleotide, a protein, or a polypeptide) is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In some embodiments, an isolated nucleic acid is preferably excised from the chromosome in which it may be found. Isolated nucleic acid molecules can be inserted into plasmids, cosmids, artificial chromosomes, and the like. Thus, in some embodiments, a recombinant nucleic acid is an isolated nucleic acid. An isolated protein may be associated with other proteins or nucleic acids, or both, with which it associates in the cell, or with cellular membranes if it is a membrane-associated protein. In some embodiments, an isolated material can be purified.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling.

Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, alkaline phosphatase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon. A fluorescent label can be used in FPIA (see, e.g., U.S. Pat. Nos. 5,593,896, 5,573,904, 5,496,925, 5,359,093, and 5,352,803, which are hereby incorporated by reference in their entireties). An acridinium compound can be used as a detectable label in a homogeneous chemiluminescent assay (see, e.g., Adamczyk et al., Bioorg. Med. Chem. Lett. 16:1324-1328 (2006); Adamczyk et al., Bioorg. Med. Chem. Lett. 4:2313-2317 (2004); Adamczyk et al., Biorg. Med. Chem. Lett. 14:3917-3921 (2004); and Adamczyk et al., Org. Lett. 5:3779-3782 (2003)).

In one aspect, the acridinium compound is an acridinium-9-carboxamide. Methods for preparing acridinium 9-carboxamides are described in Mattingly, J. Biolumin. Chemilumin. 6: 107-114 (1991); Adamczyk et al., J. Org. Chem. 63:5636-5639 (1998); Adamczyk et al., Tetrahedron 55:10899-10914 (1999); Adamczyk et al., Org. Lett. 1:779-781 (1999); Adamczyk et al., Bioconjugate Chem. 11:714-724 (2000); Mattingly et al., In Luminescence Biotechnology: Instruments and Applications; Dyke, K. V. Ed.; CRC Press: Boca Raton, pp. 77-105 (2002); Adamczyk et al., Org. Lett. 5:3779-3782 (2003); and U.S. Pat. Nos. 5,468,646, 5,543,524 and 5,783,699 (each of which is incorporated herein by reference in its entirety for its teachings regarding same).

Another example of an acridinium compound is an acridinium-9-carboxylate aryl ester. An example of an acridinium-9-carboxylate aryl ester of formula II is 10-methyl-9-(phenoxycarbonyl) acridinium fluorosulfonate (available from Cayman Chemical, Ann Arbor, MI). Methods for preparing acridinium 9-carboxylate aryl esters are described in McCapra et al., Photochem. Photobiol. 4:1111-21 (1965); Razavi et al., Luminescence 15:245-249 (2000); Razavi et al., Luminescence 15:239-244 (2000); and U.S. Pat. No. 5,241,070 (each of which is incorporated herein by reference in its entirety for its teachings regarding same). Such acridinium-9-carboxylate aryl esters are efficient chemiluminescent indicators for hydrogen peroxide produced in the oxidation of an analyte by at least one oxidase in terms of the intensity of the signal and/or the rapidity of the signal. The course of the chemiluminescent emission for the acridinium-9-carboxylate aryl ester is completed rapidly, i.e., in under 1 second, while the acridinium-9-carboxamide chemiluminescent emission extends over 2 seconds. Acridinium-9-carboxylate aryl ester, however, loses its chemiluminescent properties in the presence of protein. Therefore, its use requires the absence of protein during signal generation and detection. Methods for separating or removing proteins in the sample are well-known to those skilled in the art and include, but are not limited to, ultrafiltration, extraction, precipitation, dialysis, chromatography, and/or digestion (see, e.g., Wells, High Throughput Bioanalytical Sample Preparation. Methods and Automation Strategies, Elsevier (2003)). The amount of protein removed or separated from the test sample can be about 40%, about 45%, about 50%, about 55%, about 60%, about 65%, about 70%, about 75%, about 80%, about 85%, about 90%, or about 95%. Further details regarding acridinium-9-carboxylate aryl ester and its use are set forth in U.S. patent application Ser. No. 11/697,835, filed Apr. 9, 2007. Acridinium-9-carboxylate aryl esters can be dissolved in any suitable solvent, such as degassed anhydrous N,N-dimethylformamide (DMF) or aqueous sodium cholate.

In some embodiments, the label is alkaline phosphatase.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Linking sequences can be used for many purposes, including in recombinant Fabs. Exemplary linking sequences include, but are not limited to: (i) Histidine (His) tags, such as a 6×His tag, which has an amino acid sequence of HHHHHH (SEQ ID NO: 3), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 4) and derivatives thereof (e.g., ADDDDK (SEQ ID NO: 5), etc.); (iii) Miscellaneous sequences can be used to link or connect the light and/or heavy chain variable regions of single chain variable region fragments. Examples of other linking sequences can be found in Bird et al., Science 242:423-426(1988); Huston et al., PNAS USA 85:5879-5883 (1988); and McCafferty et al., Nature 348:552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological.

“Multivalent binding protein” is used herein to refer to a binding protein comprising two or more antigen binding sites (also referred to herein as “antigen binding domains”). A multivalent binding protein is preferably engineered to have three or more antigen binding sites and is generally not a naturally occurring antibody. The term “multispecific binding protein” refers to a binding protein that can bind two or more related or unrelated targets, including a binding protein capable of binding two or more different epitopes of the same target molecule.

“Negative predictive value” or “NPV” as used interchangeably herein refers to the probability that a subject has a negative outcome given that they have a negative test result.

“Non-point-of-care device” refers to a device that is not a point-of-care device or a single use device. A non-point-of-care device refers to any device that does not meet any of the above limitations of a point-of-care or a single use device as defined herein. In some embodiments, the non-point-of-care device may be a relatively large instrument, such as a tabletop instrument. Accordingly, in some embodiments the non-point-of-care device is not a handheld instrument. In some embodiments, the non-point-of-care device is capable of performing an assay on more than one clinical sample simultaneously. Suitable non-point-of-care devices include, for example, the Architect or Alinity platforms produced by Abbott Core Laboratories.

As used herein, the phrases “percent (%) sequence identity”, or “sequence identity” as used interchangeably herein refer to the degree of identity or correspondence between different nucleotide sequences of nucleic acid molecules (e.g., oligonucleotides, polynucleotides, etc.) or amino acid sequences of proteins that may or may not share a common evolutionary origin. Sequence identity can be determined using any publicly available sequence comparison algorithms, such as, for example, BLAST, FASTA, DNA Strider, and GCG (Genetics Computer Group, Program Manual for the GCG Package, Version 7, Madison, Wisconsin).

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

“Point-of-care device” refers to a device used to provide medical diagnostic testing at or near the point-of-care (namely, outside of a laboratory), at the time and place of patient care (such as in a hospital, physician's office, urgent or other medical care facility, a patient's home, a nursing home and/or a long-term care and/or hospice facility). Examples of point-of-care devices include those produced by Abbott Laboratories (Abbott Park, IL) (e.g., i-STAT and i-STAT Alinity, Universal Biosensors (Rowville, Australia) (see US 2006/0134713), Axis-Shield PoC AS (Oslo, Norway) and Clinical Lab Products (Los Angeles, USA).

“Polypeptide” and “protein” are used interchangeably herein and indicate a molecular chain of amino acids linked through covalent and/or noncovalent bonds. The terms do not refer to a specific length of the product. Thus, peptides, oligopeptides and proteins are included within the definition of polypeptide. The terms include post-expression modifications of the polypeptide, for example, glycosylations, acetylations, phosphorylations and the like. In addition, protein fragments, analogs, mutated or variant proteins, fusion proteins and the like are included within the meaning of polypeptide.

“Positive predictive value” or “PPV” as used interchangeably herein refers to the probability that a subject has a positive outcome given that they have a positive test result.

“Purified polypeptide” or “purified polynucleotide” refers to a polypeptide or polynucleotide of interest or fragment thereof which contains less than about 50%, less than about 70%, less than about 71%, less than about 72%, less than about 73%, less than about 74%, less than about 75%, less than about 76%, less than about 77%, less than about 78%, less than about 79%, less than about 80%, less than about 81%, less than about 82%, less than about 83%, less than about 84%, less than about 85%, less than about 86%, less than about 87%, less than about 88%, less than about 89%, or less about 90% of cellular components with which the polypeptide or polynucleotide of interest or fragment thereof is naturally associated. Methods for purifying are known in the art.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

A “receiver operating characteristic” curve or “ROC” curve refers to a graphical plot that illustrates the performance of a binary classifier system as its discrimination threshold is varied. For example, a ROC curve can be a plot of the true positive rate against the false positive rate for the different possible cutoff points of a diagnostic test. It is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate. The ROC curve demonstrates the tradeoff between sensitivity and specificity (any increase in sensitivity will be accompanied by a decrease in specificity); the closer the curve follows the left-hand border and then the top border of the ROC space, the more accurate the test; the closer the curve comes to the 45-degree diagonal of the ROC space, the less accurate the test; the slope of the tangent line at a cutoff point gives the likelihood ratio (LR) for that value of the test; and the area under the curve is a measure of test accuracy.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig®s, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

The terms “recombinant polypeptide” or “recombinant protein”, used interchangeably herein, describe a polypeptide which by virtue of its origin or manipulation is not associated with all or a portion of the polypeptide with which it is associated in nature and/or is linked to a polypeptide other than that to which it is linked in nature. A recombinant or encoded polypeptide or protein is not necessarily translated from a designated nucleic acid sequence. It also may be generated in any manner, including chemical synthesis or expression of a recombinant expression system.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative.

It is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression, so that treatment decisions regarding the subject may be made on a more informed basis.

“Sample,” “test sample,” “specimen,” “sample from a subject,” “biological sample,” and “patient sample” as used herein generally refer to a biological material being tested for and/or suspected of containing an analyte of interest, such as a Rocahepevirus sequence (e.g., a Rocahepevirus species 7). In some aspects, the sample, is suspected of containing a Rocahepevirus species 7 sample. The sample may be derived from any biological source, such as, a cervical, vaginal or anal swab or brush, or a physiological fluid including, but not limited to, whole blood, serum, plasma, interstitial fluid, saliva, ocular lens fluid, cerebral spinal fluid, sweat, urine, milk, ascites fluid, mucus, nasal fluid, sputum, synovial fluid, peritoneal fluid, vaginal fluid, menses, amniotic fluid, semen, and so forth. The sample may be used directly as obtained from the biological source or following a pretreatment to modify the character of the sample. For example, such pretreatment may include preparing plasma from blood, diluting viscous fluids, and so forth. Methods of pretreatment may also involve filtration, precipitation, dilution, distillation, mixing, concentration, lyophilization, inactivation of interfering components, the addition of reagents, lysing, etc. Moreover, it may also be beneficial to modify a solid sample to form a liquid medium or to release the analyte. Preferably, the sample may be plasma.

“Sensitivity” of an assay as used herein refers to the proportion of subjects for whom the outcome is positive that are correctly identified as positive (e.g., correctly identifying those subjects with a disease or medical condition for which they are being tested). For example, this might include correctly identifying subjects as having Rocahepevirus species 7 as distinct from those who do not have Rocahepevirus species 7.

“Specificity” of an assay as used herein refers to the proportion of subjects for whom the outcome is negative that are correctly identified as negative (e.g., correctly identifying those subjects who do not have a disease or medical condition for which they are being tested). For example, this might include correctly identifying subjects not having Rocahepevirus species 7 as distinct from those who do have a Rocahepevirus species 7.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For example, the solid phase can be plastic, derivatized plastic, magnetic, or non-magnetic metal, glass or silicon, including, for example, a test tube, microtiter well, sheet, bead, microparticle, chip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Statistically significant” as used herein refers to the likelihood that a relationship between two or more variables is caused by something other than random chance. Statistical hypothesis testing is used to determine whether the result of a data set is statistically significant. In statistical hypothesis testing, a statistically significant result is attained whenever the observed p-value of a test statistic is less than the significance level defined of the study. The p-value is the probability of obtaining results at least as extreme as those observed, given that the null hypothesis is true. Examples of statistical hypothesis analysis include Wilcoxon signed-rank test, t-test, Chi-Square or Fisher's exact test. “Significant” as used herein refers to a change that has not been determined to be statistically significant (e.g., it may not have been subject to statistical hypothesis testing).

The terms “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human. In some embodiments, the subject is a human. The subject or patient may be undergoing other forms of treatment. The subject or patient may be undergoing monitoring of the course of a bacterial infection and/or treatment of a bacterial infection. The subject or patient may have or be suspected of having a bacterial infection and/or a symptom, disease, condition, or disorder associated with the bacterial infection. In some aspects, the subject or patient may have or be suspected of having Rocahepevirus infection and one or more symptoms of hepatitis. In other aspects, the subject or patient may have or be suspected of having a Rocahepevirus infection, such as Rocahepevirus species 7 infection, and have one or more symptoms of hepatitis, such as viral hepatitis. In yet other aspects, the subject or patient may have or be suspected of having a Rocahepevirus infection, such as Rocahepevirus species 7 infection, and have one or more systems of hepatitis E virus.

“Treat,” “treating” or “treatment” are each used interchangeably herein to describe reversing, alleviating, or inhibiting the progress of a disease and/or injury, or one or more symptoms of such disease, to which such term applies. In some aspects, a treatment may be either performed in an acute or chronic way. Depending on the condition of the subject, the term also refers to preventing a disease or injury, and includes preventing the onset of a disease or injury, or preventing the symptoms associated with a disease or injury. “Preventing” also refers to preventing the recurrence of a disease or injury or of one or more symptoms associated with such disease or injury. “Treatment” and “therapeutically,” refer to the act of treating, as “treating” is defined above. In some aspects, the prevention or treatment of a disease can be done prior to affliction or injury, such as, for example, to reduce the severity of a disease or injury or symptoms associated with a disease or an injury. Such prevention or reduction can include (a) administration of one or more pharmaceutical compositions and/or one or more nutritional compositions to a subject; (b) the use of one or more of physical therapy, occupational therapy, and/or counseling; or (c) any combinations of (a) and (b).

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-UCH-L1 antibody that differs from the corresponding fragment of anti-UCH-L1 antibody in amino acid sequence but is still antigenically reactive and can compete with the corresponding fragment of anti-UCH-L1 antibody for binding with UCH-L1. “Variant” also can be used to describe a polypeptide or a fragment thereof that has been differentially processed, such as by proteolysis, phosphorylation, or other post-translational modification, yet retains its antigen reactivity.

“Vector” is used herein to describe a nucleic acid molecule that can transport another nucleic acid to which it has been linked. One type of vector is a “plasmid”, which refers to a circular double-stranded DNA loop into which additional DNA segments may be ligated. Another type of vector is a viral vector, wherein additional DNA segments may be ligated into the viral genome. Certain vectors can replicate autonomously in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. “Plasmid” and “vector” may be used interchangeably as the plasmid is the most commonly used form of vector. However, other forms of expression vectors, such as viral vectors (e.g., replication defective retroviruses, adenoviruses and adeno-associated viruses), which serve equivalent functions, can be used. In this regard, RNA versions of vectors (including RNA viral vectors) may also find use in the context of the present disclosure.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Methods of Detecting the Presence of and/or Amount or Concentration of Anti-Rocahepevirus Species 7 Antibodies or Antigen-Binding Fragments Thereof in a Biological Sample Obtained from a Subject

The present disclosure relates to methods for (a) detecting the presence of Rocahepevirus species 7 (sometimes referred to as a qualitative method or assay); or (b) determining or measuring the quantity, amount, level, or concentration (e.g., quantitating (which includes semi-quantitating)) of Rocahepevirus species 7, in one or more biological samples obtained from one or more subjects. In some aspects, the methods described herein may be (a) semi-quantitative; (b) quantitative; or (c) qualitative. An understanding of whether a method is semi-quantitative, quantitative, or qualitative, and adaptation of the method as semi-quantitative, quantitative, or qualitative is well known and done using routine techniques known in the art. In still yet other aspects, the methods described herein can be used as an aid in the diagnosis of Rocahepevirus species 7 infection. For example, the methods described herein can be used in conjunction with clinical presentation and other laboratory tests to aid in the diagnosis of Rocahepevirus species 7 infection in a subject (e.g., who may or may not exhibit signs and/or symptoms of infection and suspected of having Rocahepevirus species 7).

In some aspects, the detection in samples of antibodies directed against Rocahepevirus species 7 signals a reaction to Rocahepevirus species 7, and thus the past or current presence in the subject of Rocahepevirus species 7, such as Rocahepevirus species 7 genotype 1 or Rocahepevirus species 7 genotype 2. In one aspect, the methods relate to (a) detecting the presence of (e.g., which can be qualitative as well as semi-quantitative or quantitative) Rocahepevirus species 7 (whether genotype 1 or genotype 2) or antigen-binding fragment thereof or (b) determining or measuring the quantity, amount, level or concentration (e.g., quantitating (which includes semi-quantitating)) of anti-Rocahepevirus species 7 (whether anti-Rocahepevirus species 7 genotype 1 or Rocahepevirus species 7 genotype 2) or antigen-binding fragment thereof, in one or more biological samples obtained from one or more subjects (e.g., who may or may not exhibit signs and/or symptoms of infection and is suspected of having Rocahepevirus species 7. In some aspects, when the methods measure the quantity, amount, level or concentration (e.g., quantitating (which includes semi-quantitating)) of Rocahepevirus species 7 antibody or antigen-binding fragment thereof, the method can be performed without dilution of the biological sample. In yet another aspect, the methods relate to detecting the presence of (which can be qualitative as well as semi-quantitative or quantitative) Rocahepevirus species 7 antibody or antigen-binding fragment thereof, in one or more biological samples obtained from a subject (e.g., such as a human, a non-human primate, a cat, a dog, etc.). In another aspect, the methods relate to determining or measuring the quantity, amount, level or concentration of Rocahepevirus species 7 antibody or antigen-binding fragment thereof, in one or more biological samples obtained from a subject (e.g., such as a human, a non-human primate, a cat, a dog, etc.). In yet a further aspect, the methods relate to detecting the presence of Rocahepevirus species 7 antibody or an antigen-binding fragment thereof in one or more biological samples obtained from one or more subjects (e.g., who may or may not exhibit signs and/or symptoms of infection and suspected of having Rocahepevirus species 7).

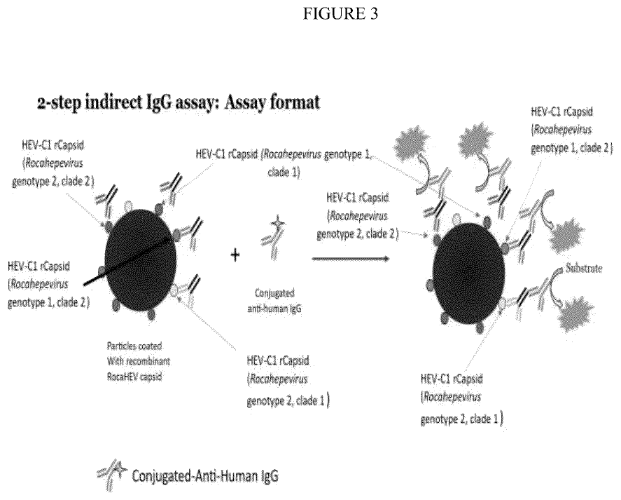

In some embodiments, detecting the presence of or measuring the quantity, amount, level or concentration of anti-Rocahepevirus species 7 antibody (such as at least one anti-Rocahepevirus species 7 genotype 1 antibody or anti-Rocahepevirus species 7 genotype 2 antibody) or antigen-binding fragment thereof includes contacting the sample, either simultaneously or sequentially, in any order, with: (1) at least at least four types of capture specific binding partners, wherein each of the four capture specific binding partners specifically binds to a Rocahepevirus species 7 antibody or an antigen-binding fragment thereof in the sample; and further wherein the four types of capture specific binding partners comprises: (i) a first capture specific binding partner (e.g., a first specific binding partner) comprising an isolated polypeptide having an amino acid sequence of SEQ ID NO:1 or a fragment thereof; (ii) a second capture specific binding partner (e.g., a second specific binding partner) comprising an isolated polypeptide having an amino acid sequence of SEQ ID NO:2 or a fragment thereof; (iii) a third capture specific binding partner (e.g., a third specific binding partner) comprising an isolated polypeptide having an amino acid sequence of SEQ ID NO:3 or a fragment thereof; and (iv) a fourth capture specific binding partner (e.g., a fourth specific binding partner) comprising an isolated polypeptide having an amino acid sequence of SEQ ID NO: 4 or a fragment thereof; and (2) at least one type of detection specific binding partner (e.g., a fifth specific binding partner) comprising at least one detectable label (e.g., detection reagent) that specifically binds to an anti-Rocahepevirus species 7 antibody or an antigen-binding fragment thereof at a different location (e.g., epitope) than the first capture specific binding partner (e.g., first specific binding partner), second capture specific binding partner (e.g., second specific binding partner), third capture specific binding partner (e.g., third specific binding partner), and the fourth capture specific binding partner (e.g., fourth specific binding partner) such that at least one type of first complex is formed comprising the four capture specific binding partners-anti-Rocahepevirus species 7 antibody-detection specific binding partner complex, and detecting the presence or determining or measuring the amount or concentration of anti-Rocahepevirus species 7 antibody or an antigen-binding fragment thereof in the sample based on the signal generated by the detectable label in the at least one type of four capture specific binding partner-anti-Rocahepevirus species 7 antibody-detection specific binding partner complex using routine techniques known in the art. An example of the method is illustrated in FIG. 3.

In yet further aspects, the above method is performed in from about 5 to about 20 minutes. In still yet further aspects, the above method is performed in less than about 20 minutes. In still yet further aspects, the above method is performed in less than about 5 minutes, less than about 10 minutes or less than about 15 minutes.

### At Least Four Types of Capture Specific Binding Partners

In some aspects, the first capture specific binding partner (e.g., first specific binding partner), the second capture specific binding partner (e.g., second specific binding partner), the third capture specific binding partner (e.g., third specific binding partner), and the fourth capture specific binding partner (e.g., fourth specific binding partner), each comprise at least one recombinant antigen. The first capture specific binding partner (e.g., first specific finding partner) comprises an isolated recombinant antigen or isolated polypeptide comprising or consisting of the amino acid sequence of SEQ ID NO:1 or a variant or a fragment thereof. The second capture specific binding partner (e.g., second specific finding partner) comprises an isolated recombinant antigen or isolated polypeptide comprising or consisting of the amino acid sequence of SEQ ID NO:2 or a variant or fragment thereof. The third capture specific binding partner (e.g., third specific finding partner) comprises an isolated recombinant antigen or isolated polypeptide comprising or consisting of the amino acid sequence of SEQ ID NO:3 or a variant or fragment thereof. The fourth capture specific binding partner (e.g., fourth specific finding partner) comprises an isolated recombinant antigen or isolated polypeptide comprising or consisting of the amino acid sequence of SEQ ID NO:4 or a variant or fragment thereof.

In some aspects, a variant recombinant antigen or polypeptide of the first capture specific binding partner, the second capture specific binding partner, the third capture specific binding partner or the fourth capture specific binding partner can one or more of substitutions and/or deletions in one or more of the following amino acid positions within SEQ ID NO:1, SEQ ID NO: 2, SEQ ID NO:3, or SEQ ID NO:4.

A “fragment” of SEQ ID NO:1 refers to an isolated protein or polypeptide that comprises or consists of a part that is less than the entirety of SEQ ID NO:11. In some aspects, a fragment of SEQ ID NO:1 can comprise or consist of 5 to 639 contiguous amino acids. In some aspects, a fragment of SEQ ID NO:1 can comprise or consist of 5 to 635 contiguous amino acids. In some aspects, a fragment of SEQ ID NO:1 can comprise or consist of 5 to 630 contiguous amino acids. In some aspects, a fragment of SEQ ID NO:1 can comprise or consist of 5 to 625 contiguous amino acids. In some aspects, a fragment of SEQ ID NO:1 can comprise or consist of 5 to 620 contiguous amino acids. In some aspects, a fragment of SEQ ID NO:1 can comprise or consist of 5 to 615 contiguous amino acids. In some aspects, a fragment of SEQ ID NO:1 can comprise or consist of 5 to 610 contiguous amino acids. In some aspects, a fragment of SEQ ID NO: 1 can comprise or consist of 5 to 605 contiguous amino acids. In some aspects, a fragment of SEQ ID NO:1 can comprise or consist of 5 to 600 contiguous amino acids. In some aspects, a fragment of SEQ ID NO:1 can comprise or consist of 5 to 595 contiguous amino acids. In some aspects, a fragment of SEQ ID NO:1 can comprise or consist of 5 to 590 contiguous amino acids. In some aspects, a fragment of SEQ ID NO:1 can comprise or consist of 5 to 585 contiguous amino acids. In some aspects, a fragment of SEQ ID NO:1 can comprise or consist of 5 to 580 contiguous amino acids. In some aspects, a fragment of SEQ ID NO:1 can comprise or consist of 5 to 575 contiguous amino acids. In some aspects, a fragment of SEQ ID NO:1 can comprise or consist of 5 to 570 contiguous amino acids. In some aspects, a fragment of SEQ ID NO:1 can comprise or consist of 5 to 565 contiguous amino acids. In some aspects, a fragment of SEQ ID NO: 1 can comprise or consist of 5 to 560 contiguous amino acids. In some aspects, a fragment of SEQ ID NO:1 can comprise or consist of 5 to 555 contiguous amino acids. In some aspects, a fragment of SEQ ID NO:1 can comprise or consist of 5 to 550 contiguous amino acids. In some aspects, a fragment of SEQ ID NO:1 can comprise or consist of 5 to 545 contiguous amino acids. In some aspects, a fragment of SEQ ID NO:1 can comprise or consist of 5 to 540 contiguous amino acids. In some aspects, a fragment of SEQ ID NO:1 can comprise or consist of 5 to 600 contiguous amino acids. In some aspects, a fragment of SEQ ID NO:1 can comprise or consist of 5 to 595 contiguous amino acids. In some aspects, a fragment of SEQ ID NO:1 can comprise or consist of 5 to 590 contiguous amino acids. In some aspects, a fragment of SEQ ID NO:1 can comprise or consist of 5 to 585 contiguous amino acids. In some aspects, a fragment of SEQ ID NO: 1 can comprise or consist of 5 to 580 contiguous amino acids. In some aspects, a fragment of SEQ ID NO:1 can comprise or consist of 5 to 575 contiguous amino acids. In some aspects, a fragment of SEQ ID NO:1 can comprise or consist of 5 to 570 contiguous amino acids. In some aspects, a fragment of SEQ ID NO:1 can comprise or consist of 5 to 565 contiguous amino acids. In some aspects, a fragment of SEQ ID NO:1 can comprise or consist of 5 to 560 contiguous amino acids. In some aspects, a fragment of SEQ ID NO:1 can comprise or consist of 5 to 555 contiguous amino acids. In some aspects, a fragment of SEQ ID NO:1 can comprise or consist of 5 to 550 contiguous amino acids. In some aspects, a fragment of SEQ ID NO:1 can comprise or consist of 5 to 545 contiguous amino acids. In some aspects, a fragment of SEQ ID NO:1 can comprise or consist of 5 to 540 contiguous amino acids. In some aspects, a fragment of SEQ ID NO: 1 can comprise or consist of 5 to 535 contiguous amino acids. In some aspects, a fragment of SEQ ID NO:1 can comprise or consist of 5 to 530 contiguous amino acids. In some aspects, a fragment of SEQ ID NO:1 can comprise or consist of 5 to 525 contiguous amino acids. In some aspects, a fragment of SEQ ID NO:1 can comprise or consist of 5 to 520 contiguous amino acids. In some aspects, a fragment of SEQ ID NO:1 can comprise or consist of 5 to 515 contiguous amino acids. In some aspects, a fragment of SEQ ID NO:1 can comprise or consist of 5 to 510 contiguous amino acids. In some aspects, a fragment of SEQ ID NO:1 can comprise or consist of 5 to 505 contiguous amino acids. In some aspects, a fragment of SEQ ID NO:1 can comprise or consist of 5 to 500 contiguous amino acids. In some aspects, a fragment of SEQ ID NO: 1 can comprise or consist of 5 to 495 contiguous amino acids. In some aspects, a fragment of SEQ ID NO:1 can comprise or consist of 5 to 490 contiguous amino acids. In some aspects, a fragment of SEQ ID NO:1 can comprise or consist of 5 to 485 contiguous amino acids. In some aspects, a fragment of SEQ ID NO:1 can comprise or consist of 5 to 480 contiguous amino acids. In some aspects, a fragment of SEQ ID NO:1 can comprise or consist of 5 to 475 contiguous amino acids. In some aspects, a fragment of SEQ ID NO:1 can comprise or consist of 5 to 470 contiguous amino acids. In some aspects, a fragment of SEQ ID NO:1 can comprise or consist of 5 to 465 contiguous amino acids. In some aspects, a fragment of SEQ ID NO:1 can comprise or consist of 5 to 460 contiguous amino acids. In some aspects, a fragment of SEQ ID NO:1 can comprise or consist of 5 to 455 contiguous amino acids. In some aspects, a fragment of SEQ ID NO: 1 can comprise or consist of 5 to 450 contiguous amino acids. In some aspects, a fragment of SEQ ID NO:1 can comprise or consist of 5 to 445 contiguous amino acids. In some aspects, a fragment of SEQ ID NO:1 can comprise or consist of 5 to 440 contiguous amino acids. In some aspects, a fragment of SEQ ID NO:1 can comprise or consist of 5 to 435 contiguous amino acids. In some aspects, a fragment of SEQ ID NO:1 can comprise or consist of 5 to 430 contiguous amino acids. In some aspects, a fragment of SEQ ID NO:1 can comprise or consist of 5 to 425 contiguous amino acids. In some aspects, a fragment of SEQ ID NO:1 can comprise or consist of 5 to 420 contiguous amino acids. In some aspects, a fragment of SEQ ID NO:1 can comprise or consist of 5 to 415 contiguous amino acids. In some aspects, a fragment of SEQ ID NO:1 can comprise or consist of 5 to 410 contiguous amino acids. In some aspects, a fragment of SEQ ID NO: 1 can comprise or consist of 5 to 405 contiguous amino acids. In some aspects, a fragment of SEQ ID NO:1 can comprise or consist of 5 to 400 contiguous amino acids. In some aspects, a fragment of SEQ ID NO:1 can comprise or consist of 5 to 395 contiguous amino acids. In some aspects, a fragment of SEQ ID NO:1 can comprise or consist of 5 to 390 contiguous amino acids. In some aspects, a fragment of SEQ ID NO:1 can comprise or consist of 5 to 385 contiguous amino acids. In some aspects, a fragment of SEQ ID NO:1 can comprise or consist of 5 to 380 contiguous amino acids. In some aspects, a fragment of SEQ ID NO:1 can comprise or consist of 5 to 375 contiguous amino acids. In some aspects, a fragment of SEQ ID NO:1 can comprise or consist of 5 to 370 contiguous amino acids. In some aspects, a fragment of SEQ ID NO:1 can comprise or consist of 5 to 365 contiguous amino acids. In some aspects, a fragment of SEQ ID NO: 1 can comprise or consist of 5 to 360 contiguous amino acids. In some aspects, a fragment of SEQ ID NO:1 can comprise or consist of 5 to 355 contiguous amino acids. In some aspects, a fragment of SEQ ID NO:1 can comprise or consist of 5 to 350 contiguous amino acids. In some aspects, a fragment of SEQ ID NO:1 can comprise or consist of 5 to 345 contiguous amino acids. In some aspects, a fragment of SEQ ID NO:1 can comprise or consist of 5 to 340 contiguous amino acids. In some aspects, a fragment of SEQ ID NO:1 can comprise or consist of 5 to 335 contiguous amino acids. In some aspects, a fragment of SEQ ID NO:1 can comprise or consist of 5 to 330 contiguous amino acids. In some aspects, a fragment of SEQ ID NO:1 can comprise or consist of 5 to 325 contiguous amino acids. In some aspects, a fragment of SEQ ID NO:1 can comprise or consist of 5 to 320 contiguous amino acids. In some aspects, a fragment of SEQ ID NO: 1 can comprise or consist of 5 to 315 contiguous amino acids. In some aspects, a fragment of SEQ ID NO:1 can comprise or consist of 5 to 310 contiguous amino acids. In some aspects, a fragment of SEQ ID NO:1 can comprise or consist of 5 to 305 contiguous amino acids. In some aspects, a fragment of SEQ ID NO:1 can comprise or consist of 5 to 300 contiguous amino acids. In some aspects, a fragment of SEQ ID NO:1 can comprise or consist of 5 to 295 contiguous amino acids. In some aspects, a fragment of SEQ ID NO:1 can comprise or consist of 5 to 290 contiguous amino acids. In some aspects, a fragment of SEQ ID NO:1 can comprise or consist of 5 to 285 contiguous amino acids. In some aspects, a fragment of SEQ ID NO:1 can comprise or consist of 5 to 280 contiguous amino acids. In some aspects, a fragment of SEQ ID NO:1 can comprise or consist of 5 to 275 contiguous amino acids. In some aspects, a fragment of SEQ ID NO: 1 can comprise or consist of 5 to 270 contiguous amino acids. In some aspects, a fragment of SEQ ID NO:1 can comprise or consist of 5 to 265 contiguous amino acids. In some aspects, a fragment of SEQ ID NO:1 can comprise or consist of 5 to 260 contiguous amino acids. In some aspects, a fragment of SEQ ID NO:1 can comprise or consist of 5 to 255 contiguous amino acids. In some aspects, a fragment of SEQ ID NO:1 can comprise or consist of 5 to 250 contiguous amino acids. In some aspects, a fragment of SEQ ID NO:1 can comprise or consist of 5 to 245 contiguous amino acids. In some aspects, a fragment of SEQ ID NO:1 can comprise or consist of 5 to 240 contiguous amino acids. In some aspects, a fragment of SEQ ID NO:1 can comprise or consist of 5 to 235 contiguous amino acids. In some aspects, a fragment of SEQ ID NO:1 can comprise or consist of 5 to 230 contiguous amino acids. In some aspects, a fragment of SEQ ID NO: 1 can comprise or consist of 5 to 225 contiguous amino acids. In some aspects, a fragment of SEQ ID NO:1 can comprise or consist of 5 to 220 contiguous amino acids. In some aspects, a fragment of SEQ ID NO:1 can comprise or consist of 5 to 215 contiguous amino acids. In some aspects, a fragment of SEQ ID NO:1 can comprise or consist of 5 to 210 contiguous amino acids. In some aspects, a fragment of SEQ ID NO:1 can comprise or consist of 5 to 205 contiguous amino acids. In some aspects, a fragment of SEQ ID NO:1 can comprise or consist of 5 to 200 contiguous amino acids. In some aspects, a fragment of SEQ ID NO:1 can comprise or consist of 5 to 195 contiguous amino acids. In some aspects, a fragment of SEQ ID NO:1 can comprise or consist of 5 to 190 contiguous amino acids. In some aspects, a fragment of SEQ ID NO:1 can comprise or consist of 5 to 185 contiguous amino acids. In some aspects, a fragment of SEQ ID NO: 1 can comprise or consist of 5 to 180 contiguous amino acids. In some aspects, a fragment of SEQ ID NO:1 can comprise or consist of 5 to 175 contiguous amino acids. In some aspects, a fragment of SEQ ID NO:1 can comprise or consist of 5 to 170 contiguous amino acids. In some aspects, a fragment of SEQ ID NO:1 can comprise or consist of 5 to 165 contiguous amino acids. In some aspects, a fragment of SEQ ID NO:1 can comprise or consist of 5 to 160 contiguous amino acids. In some aspects, a fragment of SEQ ID NO:1 can comprise or consist of 5 to 155 contiguous amino acids. In some aspects, a fragment of SEQ ID NO:1 can comprise or consist of 5 to 150 contiguous amino acids. In some aspects, a fragment of SEQ ID NO:1 can comprise or consist of 5 to 145 contiguous amino acids. In some aspects, a fragment of SEQ ID NO:1 can comprise or consist of 5 to 140 contiguous amino acids. In some aspects, a fragment of SEQ ID NO: 1 can comprise or consist of 5 to 135 contiguous amino acids. In some aspects, a fragment of SEQ ID NO:1 can comprise or consist of 5 to 130 contiguous amino acids. In some aspects, a fragment of SEQ ID NO:1 can comprise or consist of 5 to 125 contiguous amino acids. In some aspects, a fragment of SEQ ID NO:1 can comprise or consist of 5 to 120 contiguous amino acids. In some aspects, a fragment of SEQ ID NO:1 can comprise or consist of 5 to 115 contiguous amino acids. In some aspects, a fragment of SEQ ID NO:1 can comprise or consist of 5 to 110 contiguous amino acids. In some aspects, a fragment of SEQ ID NO:1 can comprise or consist of 5 to 105 contiguous amino acids. In some aspects, a fragment of SEQ ID NO:1 can comprise or consist of 5 to 100 contiguous amino acids. In some aspects, a fragment of SEQ ID NO:1 can comprise or consist of 5 to 95 contiguous amino acids. In some aspects, a fragment of SEQ ID NO: 1 can comprise or consist of 5 to 90 contiguous amino acids. In some aspects, a fragment of SEQ ID NO:1 can comprise or consist of 5 to 85 contiguous amino acids. In some aspects, a fragment of SEQ ID NO:1 can comprise or consist of 5 to 80 contiguous amino acids. In some aspects, a fragment of SEQ ID NO:1 can comprise or consist of 5 to 75 contiguous amino acids. In some aspects, a fragment of SEQ ID NO:1 can comprise or consist of 5 to 70 contiguous amino acids. In some aspects, a fragment of SEQ ID NO:1 can comprise or consist of 5 to 65 contiguous amino acids. In some aspects, a fragment of SEQ ID NO:1 can comprise or consist of 5 to 60 contiguous amino acids. In some aspects, a fragment of SEQ ID NO:1 can comprise or consist of 5 to 55 contiguous amino acids. In some aspects, a fragment of SEQ ID NO:1 can comprise or consist of 5 to 50 contiguous amino acids. In some aspects, a fragment of SEQ ID NO: 1 can comprise or consist of 5 to 45 contiguous amino acids. In some aspects, a fragment of SEQ ID NO:1 can comprise or consist of 5 to 40 contiguous amino acids. In some aspects, a fragment of SEQ ID NO:1 can comprise or consist of 5 to 35 contiguous amino acids. In some aspects, a fragment of SEQ ID NO:1 can comprise or consist of 5 to 30 contiguous amino acids. In some aspects, a fragment of SEQ ID NO:1 can comprise or consist of 5 to 25 contiguous amino acids. In some aspects, a fragment of SEQ ID NO:1 can comprise or consist of 5 to 20 contiguous amino acids. In some aspects, a fragment of SEQ ID NO:1 can comprise or consist of 5 to 15 contiguous amino acids. In some aspects, a fragment of SEQ ID NO:1 comprises or consists of SEQ ID NO:5.

A “fragment” of SEQ ID NO:2 refers to an isolated protein or polypeptide that comprises or consists of a part that is less than the entirety of SEQ ID NO:2. In some aspects, a fragment of SEQ ID NO:2 can comprise or consist of 5 to 639 contiguous amino acids. In some aspects, a fragment of SEQ ID NO:2 can comprise or consist of 5 to 635 contiguous amino acids. In some aspects, a fragment of SEQ ID NO:2 can comprise or consist of 5 to 630 contiguous amino acids. In some aspects, a fragment of SEQ ID NO:2 can comprise or consist of 5 to 625 contiguous amino acids. In some aspects, a fragment of SEQ ID NO:2 can comprise or consist of 5 to 620 contiguous amino acids. In some aspects, a fragment of SEQ ID NO:2 can comprise or consist of 5 to 615 contiguous amino acids. In some aspects, a fragment of SEQ ID NO:2 can comprise or consist of 5 to 610 contiguous amino acids. In some aspects, a fragment of SEQ ID NO: 2 can comprise or consist of 5 to 605 contiguous amino acids. In some aspects, a fragment of SEQ ID NO:2 can comprise or consist of 5 to 600 contiguous amino acids. In some aspects, a fragment of SEQ ID NO:2 can comprise or consist of 5 to 595 contiguous amino acids. In some aspects, a fragment of SEQ ID NO:2 can comprise or consist of 5 to 590 contiguous amino acids. In some aspects, a fragment of SEQ ID NO:2 can comprise or consist of 5 to 585 contiguous amino acids. In some aspects, a fragment of SEQ ID NO:2 can comprise or consist of 5 to 580 contiguous amino acids. In some aspects, a fragment of SEQ ID NO:2 can comprise or consist of 5 to 575 contiguous amino acids. In some aspects, a fragment of SEQ ID NO:2 can comprise or consist of 5 to 570 contiguous amino acids. In some aspects, a fragment of SEQ ID NO:2 can comprise or consist of 5 to 565 contiguous amino acids. In some aspects, a fragment of SEQ ID NO: 2 can comprise or consist of 5 to 560 contiguous amino acids. In some aspects, a fragment of SEQ ID NO:2 can comprise or consist of 5 to 555 contiguous amino acids. In some aspects, a fragment of SEQ ID NO:2 can comprise or consist of 5 to 550 contiguous amino acids. In some aspects, a fragment of SEQ ID NO:2 can comprise or consist of 5 to 545 contiguous amino acids. In some aspects, a fragment of SEQ ID NO:2 can comprise or consist of 5 to 540 contiguous amino acids. In some aspects, a fragment of SEQ ID NO:2 can comprise or consist of 5 to 600 contiguous amino acids. In some aspects, a fragment of SEQ ID NO:2 can comprise or consist of 5 to 595 contiguous amino acids. In some aspects, a fragment of SEQ ID NO:2 can comprise or consist of 5 to 590 contiguous amino acids. In some aspects, a fragment of SEQ ID NO:2 can comprise or consist of 5 to 585 contiguous amino acids. In some aspects, a fragment of SEQ ID NO: 2 can comprise or consist of 5 to 580 contiguous amino acids. In some aspects, a fragment of SEQ ID NO:2 can comprise or consist of 5 to 575 contiguous amino acids. In some aspects, a fragment of SEQ ID NO:2 can comprise or consist of 5 to 570 contiguous amino acids. In some aspects, a fragment of SEQ ID NO:2 can comprise or consist of 5 to 565 contiguous amino acids. In some aspects, a fragment of SEQ ID NO:2 can comprise or consist of 5 to 560 contiguous amino acids. In some aspects, a fragment of SEQ ID NO:2 can comprise or consist of 5 to 555 contiguous amino acids. In some aspects, a fragment of SEQ ID NO:2 can comprise or consist of 5 to 550 contiguous amino acids. In some aspects, a fragment of SEQ ID NO:2 can comprise or consist of 5 to 545 contiguous amino acids. In some aspects, a fragment of SEQ ID NO:2 can comprise or consist of 5 to 540 contiguous amino acids. In some aspects, a fragment of SEQ ID NO: 2 can comprise or consist of 5 to 535 contiguous amino acids. In some aspects, a fragment of SEQ ID NO:2 can comprise or consist of 5 to 530 contiguous amino acids. In some aspects, a fragment of SEQ ID NO:2 can comprise or consist of 5 to 525 contiguous amino acids. In some aspects, a fragment of SEQ ID NO:2 can comprise or consist of 5 to 520 contiguous amino acids. In some aspects, a fragment of SEQ ID NO:2 can comprise or consist of 5 to 515 contiguous amino acids. In some aspects, a fragment of SEQ ID NO:2 can comprise or consist of 5 to 510 contiguous amino acids. In some aspects, a fragment of SEQ ID NO:2 can comprise or consist of 5 to 505 contiguous amino acids. In some aspects, a fragment of SEQ ID NO:2 can comprise or consist of 5 to 500 contiguous amino acids. In some aspects, a fragment of SEQ ID NO: 2 can comprise or consist of 5 to 495 contiguous amino acids. In some aspects, a fragment of SEQ ID NO:2 can comprise or consist of 5 to 490 contiguous amino acids. In some aspects, a fragment of SEQ ID NO:2 can comprise or consist of 5 to 485 contiguous amino acids. In some aspects, a fragment of SEQ ID NO:2 can comprise or consist of 5 to 480 contiguous amino acids. In some aspects, a fragment of SEQ ID NO:2 can comprise or consist of 5 to 475 contiguous amino acids. In some aspects, a fragment of SEQ ID NO:2 can comprise or consist of 5 to 470 contiguous amino acids. In some aspects, a fragment of SEQ ID NO:2 can comprise or consist of 5 to 465 contiguous amino acids. In some aspects, a fragment of SEQ ID NO:2 can comprise or consist of 5 to 460 contiguous amino acids. In some aspects, a fragment of SEQ ID NO:2 can comprise or consist of 5 to 455 contiguous amino acids. In some aspects, a fragment of SEQ ID NO: 2 can comprise or consist of 5 to 450 contiguous amino acids. In some aspects, a fragment of SEQ ID NO:2 can comprise or consist of 5 to 445 contiguous amino acids. In some aspects, a fragment of SEQ ID NO:2 can comprise or consist of 5 to 440 contiguous amino acids. In some aspects, a fragment of SEQ ID NO:2 can comprise or consist of 5 to 435 contiguous amino acids. In some aspects, a fragment of SEQ ID NO:2 can comprise or consist of 5 to 430 contiguous amino acids. In some aspects, a fragment of SEQ ID NO:2 can comprise or consist of 5 to 425 contiguous amino acids. In some aspects, a fragment of SEQ ID NO:2 can comprise or consist of 5 to 420 contiguous amino acids. In some aspects, a fragment of SEQ ID NO:2 can comprise or consist of 5 to 415 contiguous amino acids. In some aspects, a fragment of SEQ ID NO:2 can comprise or consist of 5 to 410 contiguous amino acids. In some aspects, a fragment of SEQ ID NO: 2 can comprise or consist of 5 to 405 contiguous amino acids. In some aspects, a fragment of SEQ ID NO:2 can comprise or consist of 5 to 400 contiguous amino acids. In some aspects, a fragment of SEQ ID NO:2 can comprise or consist of 5 to 395 contiguous amino acids. In some aspects, a fragment of SEQ ID NO:2 can comprise or consist of 5 to 390 contiguous amino acids. In some aspects, a fragment of SEQ ID NO:2 can comprise or consist of 5 to 385 contiguous amino acids. In some aspects, a fragment of SEQ ID NO:2 can comprise or consist of 5 to 380 contiguous amino acids. In some aspects, a fragment of SEQ ID NO:2 can comprise or consist of 5 to 375 contiguous amino acids. In some aspects, a fragment of SEQ ID NO:2 can comprise or consist of 5 to 370 contiguous amino acids. In some aspects, a fragment of SEQ ID NO:2 can comprise or consist of 5 to 365 contiguous amino acids. In some aspects, a fragment of SEQ ID NO: 2 can comprise or consist of 5 to 360 contiguous amino acids. In some aspects, a fragment of SEQ ID NO:2 can comprise or consist of 5 to 355 contiguous amino acids. In some aspects, a fragment of SEQ ID NO:2 can comprise or consist of 5 to 350 contiguous amino acids. In some aspects, a fragment of SEQ ID NO:2 can comprise or consist of 5 to 345 contiguous amino acids. In some aspects, a fragment of SEQ ID NO:2 can comprise or consist of 5 to 340 contiguous amino acids. In some aspects, a fragment of SEQ ID NO:2 can comprise or consist of 5 to 335 contiguous amino acids. In some aspects, a fragment of SEQ ID NO:2 can comprise or consist of 5 to 330 contiguous amino acids. In some aspects, a fragment of SEQ ID NO:2 can comprise or consist of 5 to 325 contiguous amino acids. In some aspects, a fragment of SEQ ID NO:2 can comprise or consist of 5 to 320 contiguous amino acids. In some aspects, a fragment of SEQ ID NO: 2 can comprise or consist of 5 to 315 contiguous amino acids. In some aspects, a fragment of SEQ ID NO:2 can comprise or consist of 5 to 310 contiguous amino acids. In some aspects, a fragment of SEQ ID NO:2 can comprise or consist of 5 to 305 contiguous amino acids. In some aspects, a fragment of SEQ ID NO:2 can comprise or consist of 5 to 300 contiguous amino acids. In some aspects, a fragment of SEQ ID NO:2 can comprise or consist of 5 to 295 contiguous amino acids. In some aspects, a fragment of SEQ ID NO:2 can comprise or consist of 5 to 290 contiguous amino acids. In some aspects, a fragment of SEQ ID NO:2 can comprise or consist of 5 to 285 contiguous amino acids. In some aspects, a fragment of SEQ ID NO:2 can comprise or consist of 5 to 280 contiguous amino acids. In some aspects, a fragment of SEQ ID NO:2 can comprise or consist of 5 to 275 contiguous amino acids. In some aspects, a fragment of SEQ ID NO: 2 can comprise or consist of 5 to 270 contiguous amino acids. In some aspects, a fragment of SEQ ID NO:2 can comprise or consist of 5 to 265 contiguous amino acids. In some aspects, a fragment of SEQ ID NO:2 can comprise or consist of 5 to 260 contiguous amino acids. In some aspects, a fragment of SEQ ID NO:2 can comprise or consist of 5 to 255 contiguous amino acids. In some aspects, a fragment of SEQ ID NO:2 can comprise or consist of 5 to 250 contiguous amino acids. In some aspects, a fragment of SEQ ID NO:2 can comprise or consist of 5 to 245 contiguous amino acids. In some aspects, a fragment of SEQ ID NO:2 can comprise or consist of 5 to 240 contiguous amino acids. In some aspects, a fragment of SEQ ID NO:2 can comprise or consist of 5 to 235 contiguous amino acids. In some aspects, a fragment of SEQ ID NO:2 can comprise or consist of 5 to 230 contiguous amino acids. In some aspects, a fragment of SEQ ID NO: 2 can comprise or consist of 5 to 225 contiguous amino acids. In some aspects, a fragment of SEQ ID NO:2 can comprise or consist of 5 to 220 contiguous amino acids. In some aspects, a fragment of SEQ ID NO:2 can comprise or consist of 5 to 215 contiguous amino acids. In some aspects, a fragment of SEQ ID NO:2 can comprise or consist of 5 to 210 contiguous amino acids. In some aspects, a fragment of SEQ ID NO:2 can comprise or consist of 5 to 205 contiguous amino acids. In some aspects, a fragment of SEQ ID NO:2 can comprise or consist of 5 to 200 contiguous amino acids. In some aspects, a fragment of SEQ ID NO:2 can comprise or consist of 5 to 195 contiguous amino acids. In some aspects, a fragment of SEQ ID NO:2 can comprise or consist of 5 to 190 contiguous amino acids. In some aspects, a fragment of SEQ ID NO:2 can comprise or consist of 5 to 185 contiguous amino acids. In some aspects, a fragment of SEQ ID NO: 2 can comprise or consist of 5 to 180 contiguous amino acids. In some aspects, a fragment of SEQ ID NO:2 can comprise or consist of 5 to 175 contiguous amino acids. In some aspects, a fragment of SEQ ID NO:2 can comprise or consist of 5 to 170 contiguous amino acids. In some aspects, a fragment of SEQ ID NO:2 can comprise or consist of 5 to 165 contiguous amino acids. In some aspects, a fragment of SEQ ID NO:2 can comprise or consist of 5 to 160 contiguous amino acids. In some aspects, a fragment of SEQ ID NO:2 can comprise or consist of 5 to 155 contiguous amino acids. In some aspects, a fragment of SEQ ID NO:2 can comprise or consist of 5 to 150 contiguous amino acids. In some aspects, a fragment of SEQ ID NO:2 can comprise or consist of 5 to 145 contiguous amino acids. In some aspects, a fragment of SEQ ID NO:2 can comprise or consist of 5 to 140 contiguous amino acids. In some aspects, a fragment of SEQ ID NO: 2 can comprise or consist of 5 to 135 contiguous amino acids. In some aspects, a fragment of SEQ ID NO:2 can comprise or consist of 5 to 130 contiguous amino acids. In some aspects, a fragment of SEQ ID NO:2 can comprise or consist of 5 to 125 contiguous amino acids. In some aspects, a fragment of SEQ ID NO:2 can comprise or consist of 5 to 120 contiguous amino acids. In some aspects, a fragment of SEQ ID NO:2 can comprise or consist of 5 to 115 contiguous amino acids. In some aspects, a fragment of SEQ ID NO:2 can comprise or consist of 5 to 110 contiguous amino acids. In some aspects, a fragment of SEQ ID NO:2 can comprise or consist of 5 to 105 contiguous amino acids. In some aspects, a fragment of SEQ ID NO:2 can comprise or consist of 5 to 100 contiguous amino acids. In some aspects, a fragment of SEQ ID NO:2 can comprise or consist of 5 to 95 contiguous amino acids. In some aspects, a fragment of SEQ ID NO: 2 can comprise or consist of 5 to 90 contiguous amino acids. In some aspects, a fragment of SEQ ID NO:2 can comprise or consist of 5 to 85 contiguous amino acids. In some aspects, a fragment of SEQ ID NO:2 can comprise or consist of 5 to 80 contiguous amino acids. In some aspects, a fragment of SEQ ID NO:2 can comprise or consist of 5 to 75 contiguous amino acids. In some aspects, a fragment of SEQ ID NO:2 can comprise or consist of 5 to 70 contiguous amino acids. In some aspects, a fragment of SEQ ID NO:2 can comprise or consist of 5 to 65 contiguous amino acids. In some aspects, a fragment of SEQ ID NO:2 can comprise or consist of 5 to 60 contiguous amino acids. In some aspects, a fragment of SEQ ID NO:2 can comprise or consist of 5 to 55 contiguous amino acids. In some aspects, a fragment of SEQ ID NO:2 can comprise or consist of 5 to 50 contiguous amino acids. In some aspects, a fragment of SEQ ID NO: 2 can comprise or consist of 5 to 45 contiguous amino acids. In some aspects, a fragment of SEQ ID NO:2 can comprise or consist of 5 to 40 contiguous amino acids. In some aspects, a fragment of SEQ ID NO:2 can comprise or consist of 5 to 35 contiguous amino acids. In some aspects, a fragment of SEQ ID NO:2 can comprise or consist of 5 to 30 contiguous amino acids. In some aspects, a fragment of SEQ ID NO:2 can comprise or consist of 5 to 25 contiguous amino acids. In some aspects, a fragment of SEQ ID NO:2 can comprise or consist of 5 to 20 contiguous amino acids. In some aspects, a fragment of SEQ ID NO:2 can comprise or consist of 5 to 15 contiguous amino acids. In some aspects, a fragment of SEQ ID NO:2 comprises or consists of SEQ ID NO:6.

A “fragment” of SEQ ID NO:3 refers to an isolated protein or polypeptide that comprises or consists of a part that is less than the entirety of SEQ ID NO:3. In some aspects, a fragment of SEQ ID NO:3 can comprise or consist of 5 to 639 contiguous amino acids. In some aspects, a fragment of SEQ ID NO:3 can comprise or consist of 5 to 635 contiguous amino acids. In some aspects, a fragment of SEQ ID NO:3 can comprise or consist of 5 to 630 contiguous amino acids. In some aspects, a fragment of SEQ ID NO:3 can comprise or consist of 5 to 625 contiguous amino acids. In some aspects, a fragment of SEQ ID NO:3 can comprise or consist of 5 to 620 contiguous amino acids. In some aspects, a fragment of SEQ ID NO:3 can comprise or consist of 5 to 615 contiguous amino acids. In some aspects, a fragment of SEQ ID NO:3 can comprise or consist of 5 to 610 contiguous amino acids. In some aspects, a fragment of SEQ ID NO: 3 can comprise or consist of 5 to 605 contiguous amino acids. In some aspects, a fragment of SEQ ID NO:3 can comprise or consist of 5 to 600 contiguous amino acids. In some aspects, a fragment of SEQ ID NO:3 can comprise or consist of 5 to 595 contiguous amino acids. In some aspects, a fragment of SEQ ID NO:3 can comprise or consist of 5 to 590 contiguous amino acids. In some aspects, a fragment of SEQ ID NO:3 can comprise or consist of 5 to 585 contiguous amino acids. In some aspects, a fragment of SEQ ID NO:3 can comprise or consist of 5 to 580 contiguous amino acids. In some aspects, a fragment of SEQ ID NO:3 can comprise or consist of 5 to 575 contiguous amino acids. In some aspects, a fragment of SEQ ID NO:3 can comprise or consist of 5 to 570 contiguous amino acids. In some aspects, a fragment of SEQ ID NO:3 can comprise or consist of 5 to 565 contiguous amino acids. In some aspects, a fragment of SEQ ID NO: 3 can comprise or consist of 5 to 560 contiguous amino acids. In some aspects, a fragment of SEQ ID NO:3 can comprise or consist of 5 to 555 contiguous amino acids. In some aspects, a fragment of SEQ ID NO:3 can comprise or consist of 5 to 550 contiguous amino acids. In some aspects, a fragment of SEQ ID NO:3 can comprise or consist of 5 to 545 contiguous amino acids. In some aspects, a fragment of SEQ ID NO:3 can comprise or consist of 5 to 540 contiguous amino acids. In some aspects, a fragment of SEQ ID NO:3 can comprise or consist of 5 to 600 contiguous amino acids. In some aspects, a fragment of SEQ ID NO:3 can comprise or consist of 5 to 595 contiguous amino acids. In some aspects, a fragment of SEQ ID NO:3 can comprise or consist of 5 to 590 contiguous amino acids. In some aspects, a fragment of SEQ ID NO:3 can comprise or consist of 5 to 585 contiguous amino acids. In some aspects, a fragment of SEQ ID NO: 3 can comprise or consist of 5 to 580 contiguous amino acids. In some aspects, a fragment of SEQ ID NO:3 can comprise or consist of 5 to 575 contiguous amino acids. In some aspects, a fragment of SEQ ID NO:3 can comprise or consist of 5 to 570 contiguous amino acids. In some aspects, a fragment of SEQ ID NO:3 can comprise or consist of 5 to 565 contiguous amino acids. In some aspects, a fragment of SEQ ID NO:3 can comprise or consist of 5 to 560 contiguous amino acids. In some aspects, a fragment of SEQ ID NO:3 can comprise or consist of 5 to 555 contiguous amino acids. In some aspects, a fragment of SEQ ID NO:3 can comprise or consist of 5 to 550 contiguous amino acids. In some aspects, a fragment of SEQ ID NO:3 can comprise or consist of 5 to 545 contiguous amino acids. In some aspects, a fragment of SEQ ID NO:3 can comprise or consist of 5 to 540 contiguous amino acids. In some aspects, a fragment of SEQ ID NO: 3 can comprise or consist of 5 to 535 contiguous amino acids. In some aspects, a fragment of SEQ ID NO:3 can comprise or consist of 5 to 530 contiguous amino acids. In some aspects, a fragment of SEQ ID NO:3 can comprise or consist of 5 to 525 contiguous amino acids. In some aspects, a fragment of SEQ ID NO:3 can comprise or consist of 5 to 520 contiguous amino acids. In some aspects, a fragment of SEQ ID NO:3 can comprise or consist of 5 to 515 contiguous amino acids. In some aspects, a fragment of SEQ ID NO:3 can comprise or consist of 5 to 510 contiguous amino acids. In some aspects, a fragment of SEQ ID NO:3 can comprise or consist of 5 to 505 contiguous amino acids. In some aspects, a fragment of SEQ ID NO:3 can comprise or consist of 5 to 500 contiguous amino acids. In some aspects, a fragment of SEQ ID NO: 3 can comprise or consist of 5 to 495 contiguous amino acids. In some aspects, a fragment of SEQ ID NO:3 can comprise or consist of 5 to 490 contiguous amino acids. In some aspects, a fragment of SEQ ID NO:3 can comprise or consist of 5 to 485 contiguous amino acids. In some aspects, a fragment of SEQ ID NO:3 can comprise or consist of 5 to 480 contiguous amino acids. In some aspects, a fragment of SEQ ID NO:3 can comprise or consist of 5 to 475 contiguous amino acids. In some aspects, a fragment of SEQ ID NO:3 can comprise or consist of 5 to 470 contiguous amino acids. In some aspects, a fragment of SEQ ID NO:3 can comprise or consist of 5 to 465 contiguous amino acids. In some aspects, a fragment of SEQ ID NO:3 can comprise or consist of 5 to 460 contiguous amino acids. In some aspects, a fragment of SEQ ID NO:3 can comprise or consist of 5 to 455 contiguous amino acids. In some aspects, a fragment of SEQ ID NO: 3 can comprise or consist of 5 to 450 contiguous amino acids. In some aspects, a fragment of SEQ ID NO:3 can comprise or consist of 5 to 445 contiguous amino acids. In some aspects, a fragment of SEQ ID NO:3 can comprise or consist of 5 to 440 contiguous amino acids. In some aspects, a fragment of SEQ ID NO:3 can comprise or consist of 5 to 435 contiguous amino acids. In some aspects, a fragment of SEQ ID NO:3 can comprise or consist of 5 to 430 contiguous amino acids. In some aspects, a fragment of SEQ ID NO:3 can comprise or consist of 5 to 425 contiguous amino acids. In some aspects, a fragment of SEQ ID NO:3 can comprise or consist of 5 to 420 contiguous amino acids. In some aspects, a fragment of SEQ ID NO:3 can comprise or consist of 5 to 415 contiguous amino acids. In some aspects, a fragment of SEQ ID NO:3 can comprise or consist of 5 to 410 contiguous amino acids. In some aspects, a fragment of SEQ ID NO: 3 can comprise or consist of 5 to 405 contiguous amino acids. In some aspects, a fragment of SEQ ID NO:3 can comprise or consist of 5 to 400 contiguous amino acids. In some aspects, a fragment of SEQ ID NO:3 can comprise or consist of 5 to 395 contiguous amino acids. In some aspects, a fragment of SEQ ID NO:3 can comprise or consist of 5 to 390 contiguous amino acids. In some aspects, a fragment of SEQ ID NO:3 can comprise or consist of 5 to 385 contiguous amino acids. In some aspects, a fragment of SEQ ID NO:3 can comprise or consist of 5 to 380 contiguous amino acids. In some aspects, a fragment of SEQ ID NO:3 can comprise or consist of 5 to 375 contiguous amino acids. In some aspects, a fragment of SEQ ID NO:3 can comprise or consist of 5 to 370 contiguous amino acids. In some aspects, a fragment of SEQ ID NO:3 can comprise or consist of 5 to 365 contiguous amino acids. In some aspects, a fragment of SEQ ID NO: 3 can comprise or consist of 5 to 360 contiguous amino acids. In some aspects, a fragment of SEQ ID NO:3 can comprise or consist of 5 to 355 contiguous amino acids. In some aspects, a fragment of SEQ ID NO:3 can comprise or consist of 5 to 350 contiguous amino acids. In some aspects, a fragment of SEQ ID NO:3 can comprise or consist of 5 to 345 contiguous amino acids. In some aspects, a fragment of SEQ ID NO:3 can comprise or consist of 5 to 340 contiguous amino acids. In some aspects, a fragment of SEQ ID NO:3 can comprise or consist of 5 to 335 contiguous amino acids. In some aspects, a fragment of SEQ ID NO:3 can comprise or consist of 5 to 330 contiguous amino acids. In some aspects, a fragment of SEQ ID NO:3 can comprise or consist of 5 to 325 contiguous amino acids. In some aspects, a fragment of SEQ ID NO:3 can comprise or consist of 5 to 320 contiguous amino acids. In some aspects, a fragment of SEQ ID NO: 3 can comprise or consist of 5 to 315 contiguous amino acids. In some aspects, a fragment of SEQ ID NO:3 can comprise or consist of 5 to 310 contiguous amino acids. In some aspects, a fragment of SEQ ID NO:3 can comprise or consist of 5 to 305 contiguous amino acids. In some aspects, a fragment of SEQ ID NO:3 can comprise or consist of 5 to 300 contiguous amino acids. In some aspects, a fragment of SEQ ID NO:3 can comprise or consist of 5 to 295 contiguous amino acids. In some aspects, a fragment of SEQ ID NO:3 can comprise or consist of 5 to 290 contiguous amino acids. In some aspects, a fragment of SEQ ID NO:3 can comprise or consist of 5 to 285 contiguous amino acids. In some aspects, a fragment of SEQ ID NO:3 can comprise or consist of 5 to 280 contiguous amino acids. In some aspects, a fragment of SEQ ID NO:3 can comprise or consist of 5 to 275 contiguous amino acids. In some aspects, a fragment of SEQ ID NO: 3 can comprise or consist of 5 to 270 contiguous amino acids. In some aspects, a fragment of SEQ ID NO:3 can comprise or consist of 5 to 265 contiguous amino acids. In some aspects, a fragment of SEQ ID NO:3 can comprise or consist of 5 to 260 contiguous amino acids. In some aspects, a fragment of SEQ ID NO:3 can comprise or consist of 5 to 255 contiguous amino acids. In some aspects, a fragment of SEQ ID NO:3 can comprise or consist of 5 to 250 contiguous amino acids. In some aspects, a fragment of SEQ ID NO:3 can comprise or consist of 5 to 245 contiguous amino acids. In some aspects, a fragment of SEQ ID NO:3 can comprise or consist of 5 to 240 contiguous amino acids. In some aspects, a fragment of SEQ ID NO:3 can comprise or consist of 5 to 235 contiguous amino acids. In some aspects, a fragment of SEQ ID NO:3 can comprise or consist of 5 to 230 contiguous amino acids. In some aspects, a fragment of SEQ ID NO: 3 can comprise or consist of 5 to 225 contiguous amino acids. In some aspects, a fragment of SEQ ID NO:3 can comprise or consist of 5 to 220 contiguous amino acids. In some aspects, a fragment of SEQ ID NO:3 can comprise or consist of 5 to 215 contiguous amino acids. In some aspects, a fragment of SEQ ID NO:3 can comprise or consist of 5 to 210 contiguous amino acids. In some aspects, a fragment of SEQ ID NO:3 can comprise or consist of 5 to 205 contiguous amino acids. In some aspects, a fragment of SEQ ID NO:3 can comprise or consist of 5 to 200 contiguous amino acids. In some aspects, a fragment of SEQ ID NO:3 can comprise or consist of 5 to 195 contiguous amino acids. In some aspects, a fragment of SEQ ID NO:3 can comprise or consist of 5 to 190 contiguous amino acids. In some aspects, a fragment of SEQ ID NO:3 can comprise or consist of 5 to 185 contiguous amino acids. In some aspects, a fragment of SEQ ID NO: 3 can comprise or consist of 5 to 180 contiguous amino acids. In some aspects, a fragment of SEQ ID NO:3 can comprise or consist of 5 to 175 contiguous amino acids. In some aspects, a fragment of SEQ ID NO:3 can comprise or consist of 5 to 170 contiguous amino acids. In some aspects, a fragment of SEQ ID NO:3 can comprise or consist of 5 to 165 contiguous amino acids. In some aspects, a fragment of SEQ ID NO:3 can comprise or consist of 5 to 160 contiguous amino acids. In some aspects, a fragment of SEQ ID NO:3 can comprise or consist of 5 to 155 contiguous amino acids. In some aspects, a fragment of SEQ ID NO:3 can comprise or consist of 5 to 150 contiguous amino acids. In some aspects, a fragment of SEQ ID NO:3 can comprise or consist of 5 to 145 contiguous amino acids. In some aspects, a fragment of SEQ ID NO:3 can comprise or consist of 5 to 140 contiguous amino acids. In some aspects, a fragment of SEQ ID NO: 3 can comprise or consist of 5 to 135 contiguous amino acids. In some aspects, a fragment of SEQ ID NO:3 can comprise or consist of 5 to 130 contiguous amino acids. In some aspects, a fragment of SEQ ID NO:3 can comprise or consist of 5 to 125 contiguous amino acids. In some aspects, a fragment of SEQ ID NO:3 can comprise or consist of 5 to 120 contiguous amino acids. In some aspects, a fragment of SEQ ID NO:3 can comprise or consist of 5 to 115 contiguous amino acids. In some aspects, a fragment of SEQ ID NO:3 can comprise or consist of 5 to 110 contiguous amino acids. In some aspects, a fragment of SEQ ID NO:3 can comprise or consist of 5 to 105 contiguous amino acids. In some aspects, a fragment of SEQ ID NO:3 can comprise or consist of 5 to 100 contiguous amino acids. In some aspects, a fragment of SEQ ID NO:3 can comprise or consist of 5 to 95 contiguous amino acids. In some aspects, a fragment of SEQ ID NO: 3 can comprise or consist of 5 to 90 contiguous amino acids. In some aspects, a fragment of SEQ ID NO:3 can comprise or consist of 5 to 85 contiguous amino acids. In some aspects, a fragment of SEQ ID NO:3 can comprise or consist of 5 to 80 contiguous amino acids. In some aspects, a fragment of SEQ ID NO:3 can comprise or consist of 5 to 75 contiguous amino acids. In some aspects, a fragment of SEQ ID NO:3 can comprise or consist of 5 to 70 contiguous amino acids. In some aspects, a fragment of SEQ ID NO:3 can comprise or consist of 5 to 65 contiguous amino acids. In some aspects, a fragment of SEQ ID NO:3 can comprise or consist of 5 to 60 contiguous amino acids. In some aspects, a fragment of SEQ ID NO:3 can comprise or consist of 5 to 55 contiguous amino acids. In some aspects, a fragment of SEQ ID NO:3 can comprise or consist of 5 to 50 contiguous amino acids. In some aspects, a fragment of SEQ ID NO: 3 can comprise or consist of 5 to 45 contiguous amino acids. In some aspects, a fragment of SEQ ID NO:3 can comprise or consist of 5 to 40 contiguous amino acids. In some aspects, a fragment of SEQ ID NO:3 can comprise or consist of 5 to 35 contiguous amino acids. In some aspects, a fragment of SEQ ID NO:3 can comprise or consist of 5 to 30 contiguous amino acids. In some aspects, a fragment of SEQ ID NO:3 can comprise or consist of 5 to 25 contiguous amino acids. In some aspects, a fragment of SEQ ID NO:3 can comprise or consist of 5 to 20 contiguous amino acids. In some aspects, a fragment of SEQ ID NO:3 can comprise or consist of 5 to 15 contiguous amino acids. In some aspects, a fragment of SEQ ID NO:3 comprises or consists of SEQ ID NO:7.

A “fragment” of SEQ ID NO:4 refers to an isolated protein or polypeptide that comprises or consists of a part that is less than the entirety of SEQ ID NO:4. In some aspects, a fragment of SEQ ID NO:4 can comprise or consist of 5 to 639 contiguous amino acids. In some aspects, a fragment of SEQ ID NO:4 can comprise or consist of 5 to 635 contiguous amino acids. In some aspects, a fragment of SEQ ID NO:4 can comprise or consist of 5 to 630 contiguous amino acids. In some aspects, a fragment of SEQ ID NO:4 can comprise or consist of 5 to 625 contiguous amino acids. In some aspects, a fragment of SEQ ID NO:4 can comprise or consist of 5 to 620 contiguous amino acids. In some aspects, a fragment of SEQ ID NO:4 can comprise or consist of 5 to 615 contiguous amino acids. In some aspects, a fragment of SEQ ID NO:4 can comprise or consist of 5 to 610 contiguous amino acids. In some aspects, a fragment of SEQ ID NO: 4 can comprise or consist of 5 to 605 contiguous amino acids. In some aspects, a fragment of SEQ ID NO:4 can comprise or consist of 5 to 600 contiguous amino acids. In some aspects, a fragment of SEQ ID NO:4 can comprise or consist of 5 to 595 contiguous amino acids. In some aspects, a fragment of SEQ ID NO:4 can comprise or consist of 5 to 590 contiguous amino acids. In some aspects, a fragment of SEQ ID NO:4 can comprise or consist of 5 to 585 contiguous amino acids. In some aspects, a fragment of SEQ ID NO:4 can comprise or consist of 5 to 580 contiguous amino acids. In some aspects, a fragment of SEQ ID NO:4 can comprise or consist of 5 to 575 contiguous amino acids. In some aspects, a fragment of SEQ ID NO:4 can comprise or consist of 5 to 570 contiguous amino acids. In some aspects, a fragment of SEQ ID NO:4 can comprise or consist of 5 to 565 contiguous amino acids. In some aspects, a fragment of SEQ ID NO: 4 can comprise or consist of 5 to 560 contiguous amino acids. In some aspects, a fragment of SEQ ID NO:4 can comprise or consist of 5 to 555 contiguous amino acids. In some aspects, a fragment of SEQ ID NO:4 can comprise or consist of 5 to 550 contiguous amino acids. In some aspects, a fragment of SEQ ID NO:4 can comprise or consist of 5 to 545 contiguous amino acids. In some aspects, a fragment of SEQ ID NO:4 can comprise or consist of 5 to 540 contiguous amino acids. In some aspects, a fragment of SEQ ID NO:4 can comprise or consist of 5 to 600 contiguous amino acids. In some aspects, a fragment of SEQ ID NO:4 can comprise or consist of 5 to 595 contiguous amino acids. In some aspects, a fragment of SEQ ID NO:4 can comprise or consist of 5 to 590 contiguous amino acids. In some aspects, a fragment of SEQ ID NO:4 can comprise or consist of 5 to 585 contiguous amino acids. In some aspects, a fragment of SEQ ID NO: 4 can comprise or consist of 5 to 580 contiguous amino acids. In some aspects, a fragment of SEQ ID NO:4 can comprise or consist of 5 to 575 contiguous amino acids. In some aspects, a fragment of SEQ ID NO:4 can comprise or consist of 5 to 570 contiguous amino acids. In some aspects, a fragment of SEQ ID NO:4 can comprise or consist of 5 to 565 contiguous amino acids. In some aspects, a fragment of SEQ ID NO:4 can comprise or consist of 5 to 560 contiguous amino acids. In some aspects, a fragment of SEQ ID NO:4 can comprise or consist of 5 to 555 contiguous amino acids. In some aspects, a fragment of SEQ ID NO:4 can comprise or consist of 5 to 550 contiguous amino acids. In some aspects, a fragment of SEQ ID NO:4 can comprise or consist of 5 to 545 contiguous amino acids. In some aspects, a fragment of SEQ ID NO:4 can comprise or consist of 5 to 540 contiguous amino acids. In some aspects, a fragment of SEQ ID NO: 4 can comprise or consist of 5 to 535 contiguous amino acids. In some aspects, a fragment of SEQ ID NO:4 can comprise or consist of 5 to 530 contiguous amino acids. In some aspects, a fragment of SEQ ID NO:4 can comprise or consist of 5 to 525 contiguous amino acids. In some aspects, a fragment of SEQ ID NO:4 can comprise or consist of 5 to 520 contiguous amino acids. In some aspects, a fragment of SEQ ID NO:4 can comprise or consist of 5 to 515 contiguous amino acids. In some aspects, a fragment of SEQ ID NO:4 can comprise or consist of 5 to 510 contiguous amino acids. In some aspects, a fragment of SEQ ID NO:4 can comprise or consist of 5 to 505 contiguous amino acids. In some aspects, a fragment of SEQ ID NO:4 can comprise or consist of 5 to 500 contiguous amino acids. In some aspects, a fragment of SEQ ID NO: 4 can comprise or consist of 5 to 495 contiguous amino acids. In some aspects, a fragment of SEQ ID NO:4 can comprise or consist of 5 to 490 contiguous amino acids. In some aspects, a fragment of SEQ ID NO:4 can comprise or consist of 5 to 485 contiguous amino acids. In some aspects, a fragment of SEQ ID NO:4 can comprise or consist of 5 to 480 contiguous amino acids. In some aspects, a fragment of SEQ ID NO:4 can comprise or consist of 5 to 475 contiguous amino acids. In some aspects, a fragment of SEQ ID NO:4 can comprise or consist of 5 to 470 contiguous amino acids. In some aspects, a fragment of SEQ ID NO:4 can comprise or consist of 5 to 465 contiguous amino acids. In some aspects, a fragment of SEQ ID NO:4 can comprise or consist of 5 to 460 contiguous amino acids. In some aspects, a fragment of SEQ ID NO:4 can comprise or consist of 5 to 455 contiguous amino acids. In some aspects, a fragment of SEQ ID NO: 4 can comprise or consist of 5 to 450 contiguous amino acids. In some aspects, a fragment of SEQ ID NO:4 can comprise or consist of 5 to 445 contiguous amino acids. In some aspects, a fragment of SEQ ID NO:4 can comprise or consist of 5 to 440 contiguous amino acids. In some aspects, a fragment of SEQ ID NO:4 can comprise or consist of 5 to 435 contiguous amino acids. In some aspects, a fragment of SEQ ID NO:4 can comprise or consist of 5 to 430 contiguous amino acids. In some aspects, a fragment of SEQ ID NO:4 can comprise or consist of 5 to 425 contiguous amino acids. In some aspects, a fragment of SEQ ID NO:4 can comprise or consist of 5 to 420 contiguous amino acids. In some aspects, a fragment of SEQ ID NO:4 can comprise or consist of 5 to 415 contiguous amino acids. In some aspects, a fragment of SEQ ID NO:4 can comprise or consist of 5 to 410 contiguous amino acids. In some aspects, a fragment of SEQ ID NO: 4 can comprise or consist of 5 to 405 contiguous amino acids. In some aspects, a fragment of SEQ ID NO:4 can comprise or consist of 5 to 400 contiguous amino acids. In some aspects, a fragment of SEQ ID NO:4 can comprise or consist of 5 to 395 contiguous amino acids. In some aspects, a fragment of SEQ ID NO:4 can comprise or consist of 5 to 390 contiguous amino acids. In some aspects, a fragment of SEQ ID NO:4 can comprise or consist of 5 to 385 contiguous amino acids. In some aspects, a fragment of SEQ ID NO:4 can comprise or consist of 5 to 380 contiguous amino acids. In some aspects, a fragment of SEQ ID NO:4 can comprise or consist of 5 to 375 contiguous amino acids. In some aspects, a fragment of SEQ ID NO:4 can comprise or consist of 5 to 370 contiguous amino acids. In some aspects, a fragment of SEQ ID NO:4 can comprise or consist of 5 to 365 contiguous amino acids. In some aspects, a fragment of SEQ ID NO: 4 can comprise or consist of 5 to 360 contiguous amino acids. In some aspects, a fragment of SEQ ID NO:4 can comprise or consist of 5 to 355 contiguous amino acids. In some aspects, a fragment of SEQ ID NO:4 can comprise or consist of 5 to 350 contiguous amino acids. In some aspects, a fragment of SEQ ID NO:4 can comprise or consist of 5 to 345 contiguous amino acids. In some aspects, a fragment of SEQ ID NO:4 can comprise or consist of 5 to 340 contiguous amino acids. In some aspects, a fragment of SEQ ID NO:4 can comprise or consist of 5 to 335 contiguous amino acids. In some aspects, a fragment of SEQ ID NO:4 can comprise or consist of 5 to 330 contiguous amino acids. In some aspects, a fragment of SEQ ID NO:4 can comprise or consist of 5 to 325 contiguous amino acids. In some aspects, a fragment of SEQ ID NO:4 can comprise or consist of 5 to 320 contiguous amino acids. In some aspects, a fragment of SEQ ID NO: 4 can comprise or consist of 5 to 315 contiguous amino acids. In some aspects, a fragment of SEQ ID NO:4 can comprise or consist of 5 to 310 contiguous amino acids. In some aspects, a fragment of SEQ ID NO:4 can comprise or consist of 5 to 305 contiguous amino acids. In some aspects, a fragment of SEQ ID NO:4 can comprise or consist of 5 to 300 contiguous amino acids. In some aspects, a fragment of SEQ ID NO:4 can comprise or consist of 5 to 295 contiguous amino acids. In some aspects, a fragment of SEQ ID NO:4 can comprise or consist of 5 to 290 contiguous amino acids. In some aspects, a fragment of SEQ ID NO:4 can comprise or consist of 5 to 285 contiguous amino acids. In some aspects, a fragment of SEQ ID NO:4 can comprise or consist of 5 to 280 contiguous amino acids. In some aspects, a fragment of SEQ ID NO:4 can comprise or consist of 5 to 275 contiguous amino acids. In some aspects, a fragment of SEQ ID NO: 4 can comprise or consist of 5 to 270 contiguous amino acids. In some aspects, a fragment of SEQ ID NO:4 can comprise or consist of 5 to 265 contiguous amino acids. In some aspects, a fragment of SEQ ID NO:4 can comprise or consist of 5 to 260 contiguous amino acids. In some aspects, a fragment of SEQ ID NO:4 can comprise or consist of 5 to 255 contiguous amino acids. In some aspects, a fragment of SEQ ID NO:4 can comprise or consist of 5 to 250 contiguous amino acids. In some aspects, a fragment of SEQ ID NO:4 can comprise or consist of 5 to 245 contiguous amino acids. In some aspects, a fragment of SEQ ID NO:4 can comprise or consist of 5 to 240 contiguous amino acids. In some aspects, a fragment of SEQ ID NO:4 can comprise or consist of 5 to 235 contiguous amino acids. In some aspects, a fragment of SEQ ID NO:4 can comprise or consist of 5 to 230 contiguous amino acids. In some aspects, a fragment of SEQ ID NO: 4 can comprise or consist of 5 to 225 contiguous amino acids. In some aspects, a fragment of SEQ ID NO:4 can comprise or consist of 5 to 220 contiguous amino acids. In some aspects, a fragment of SEQ ID NO:4 can comprise or consist of 5 to 215 contiguous amino acids. In some aspects, a fragment of SEQ ID NO:4 can comprise or consist of 5 to 210 contiguous amino acids. In some aspects, a fragment of SEQ ID NO:4 can comprise or consist of 5 to 205 contiguous amino acids. In some aspects, a fragment of SEQ ID NO:4 can comprise or consist of 5 to 200 contiguous amino acids. In some aspects, a fragment of SEQ ID NO:4 can comprise or consist of 5 to 195 contiguous amino acids. In some aspects, a fragment of SEQ ID NO:4 can comprise or consist of 5 to 190 contiguous amino acids. In some aspects, a fragment of SEQ ID NO:4 can comprise or consist of 5 to 185 contiguous amino acids. In some aspects, a fragment of SEQ ID NO: 4 can comprise or consist of 5 to 180 contiguous amino acids. In some aspects, a fragment of SEQ ID NO:4 can comprise or consist of 5 to 175 contiguous amino acids. In some aspects, a fragment of SEQ ID NO:4 can comprise or consist of 5 to 170 contiguous amino acids. In some aspects, a fragment of SEQ ID NO:4 can comprise or consist of 5 to 165 contiguous amino acids. In some aspects, a fragment of SEQ ID NO:4 can comprise or consist of 5 to 160 contiguous amino acids. In some aspects, a fragment of SEQ ID NO:4 can comprise or consist of 5 to 155 contiguous amino acids. In some aspects, a fragment of SEQ ID NO:4 can comprise or consist of 5 to 150 contiguous amino acids. In some aspects, a fragment of SEQ ID NO:4 can comprise or consist of 5 to 145 contiguous amino acids. In some aspects, a fragment of SEQ ID NO:4 can comprise or consist of 5 to 140 contiguous amino acids. In some aspects, a fragment of SEQ ID NO: 4 can comprise or consist of 5 to 135 contiguous amino acids. In some aspects, a fragment of SEQ ID NO:4 can comprise or consist of 5 to 130 contiguous amino acids. In some aspects, a fragment of SEQ ID NO:4 can comprise or consist of 5 to 125 contiguous amino acids. In some aspects, a fragment of SEQ ID NO:4 can comprise or consist of 5 to 120 contiguous amino acids. In some aspects, a fragment of SEQ ID NO:4 can comprise or consist of 5 to 115 contiguous amino acids. In some aspects, a fragment of SEQ ID NO:4 can comprise or consist of 5 to 110 contiguous amino acids. In some aspects, a fragment of SEQ ID NO:4 can comprise or consist of 5 to 105 contiguous amino acids. In some aspects, a fragment of SEQ ID NO:4 can comprise or consist of 5 to 100 contiguous amino acids. In some aspects, a fragment of SEQ ID NO:4 can comprise or consist of 5 to 95 contiguous amino acids. In some aspects, a fragment of SEQ ID NO: 4 can comprise or consist of 5 to 90 contiguous amino acids. In some aspects, a fragment of SEQ ID NO:4 can comprise or consist of 5 to 85 contiguous amino acids. In some aspects, a fragment of SEQ ID NO:4 can comprise or consist of 5 to 80 contiguous amino acids. In some aspects, a fragment of SEQ ID NO:4 can comprise or consist of 5 to 75 contiguous amino acids. In some aspects, a fragment of SEQ ID NO:4 can comprise or consist of 5 to 70 contiguous amino acids. In some aspects, a fragment of SEQ ID NO:4 can comprise or consist of 5 to 65 contiguous amino acids. In some aspects, a fragment of SEQ ID NO:4 can comprise or consist of 5 to 60 contiguous amino acids. In some aspects, a fragment of SEQ ID NO:4 can comprise or consist of 5 to 55 contiguous amino acids. In some aspects, a fragment of SEQ ID NO:4 can comprise or consist of 5 to 50 contiguous amino acids. In some aspects, a fragment of SEQ ID NO: 4 can comprise or consist of 5 to 45 contiguous amino acids. In some aspects, a fragment of SEQ ID NO:4 can comprise or consist of 5 to 40 contiguous amino acids. In some aspects, a fragment of SEQ ID NO:4 can comprise or consist of 5 to 35 contiguous amino acids. In some aspects, a fragment of SEQ ID NO:4 can comprise or consist of 5 to 30 contiguous amino acids. In some aspects, a fragment of SEQ ID NO:4 can comprise or consist of 5 to 25 contiguous amino acids. In some aspects, a fragment of SEQ ID NO:4 can comprise or consist of 5 to 20 contiguous amino acids. In some aspects, a fragment of SEQ ID NO:4 can comprise or consist of 5 to 15 contiguous amino acids. In some aspects, a fragment of SEQ ID NO:4 comprises or consists of SEQ ID NO:8.

### At Least One Detection Specific Binding Partner

The at least one detection specific binding partner (e.g., a fifth specific binding partner) comprising at least detectable label can be either at least one recombinant antigen or at least one antibody. In some aspects, the at least one detection specific binding partner is at least one recombinant antigen which comprises all or at least a portion of at least one isolated polypeptide comprising or consisting of any of SEQ ID NO:1 or a variant or fragment thereof, SEQ ID NO:2 or a variant or fragment thereof, SEQ ID NO:3 or a variant or fragment thereof, and/or SEQ ID NO:4 or a variant or fragment thereof. The isolated polypeptides used in the recombinant antigens described previously herein for any of the four types of capture specific binding partner can also be used with the at least one detection specific binding partner (e.g., derived from the same materials, have the same lengths, etc). Moreover, the isolated polypeptides used in the recombinant antigen of the at least one detection specific binding partner can be the same or different than the isolated polypeptides used in the recombinant antigen of any of the four types of capture specific binding partners.

Alternatively, the at least one second specific binding partner can comprise at least one antibody that specifically binds to anti-Rocahepevirus species 7 antibody or an antigen-binding fragment thereof (e.g., analyte) contained in the at least one complex comprising the four types of capture specific binding partners and anti-Rocahepevirus species 7 antibody. The antibody used as the at least one detection specific binding partner (e.g, fifth specific binding partner) is not critical and can be a polyclonal antibody, a monoclonal antibody, a humanized antibody, a chimeric antibody, a fully human antibody, a bispecific antibody or a multi-specific antibody. In one aspect, the antibody used as at least one detection specific binding partner can be an anti-species IgG (e.g., anti-human-IgG IgG) antibody, an anti-species IgM (e.g., anti-human-IgM IgG) antibody or an anti-species IgG (e.g., anti-human-IgG IgG) and anti-species IgM (e.g., anti-human-IgM IgG) antibody. For example, in some aspects, the antibody used as the at least one detection specific binding partner can be a mouse IgG antibody, a mouse IgM antibody, a mouse IgG and mouse IgM antibody, a rat IgG antibody, a rat IgM antibody, a rat IgG and rat IgM antibody, a rabbit IgG antibody, a rabbit IgM antibody, a rabbit IgG and rabbit IgM antibody, a goat IgG antibody, a goat IgM antibody, a goat IgG and goat IgM antibody, a sheep IgG antibody, a sheep IgM antibody, a sheep IgG and sheep IgM antibody, a non-human primate IgG antibody, a non-human primate IgM antibody, a non-human primate IgG and non-human primate IgM antibody, a human IgG antibody, a human IgM antibody or a human IgG and human IgM antibody.

In some aspects, any or all of the capture specific binding partners or detection specific binding partners can be immobilized on a solid support. For example, in some aspects, (1) the first capture specific binding partner (e.g., first specific binding partner), can be immobilized on a solid support; (2) the second capture specific binding partner (e.g., second specific binding partner) can be immobilized on a solid support; (3) the third capture specific binding partner (e.g., third specific binding partner) can be immobilized on a solid support; (4) the fourth capture specific binding partner (e.g., fourth specific binding partner) can be immobilized on a solid support; or (5) any combination of (1)-(4). In some aspects, each of the first capture specific binding partner, second capture specific binding partner, third capture specific binding partner, and fourth capture specific binding partner are immobilized on a solid support. In some further aspects, at least one of the detection specific binding partner (e.g., fifth specific binding partner) can be immobilized on a solid support.

In some embodiments, the biological sample is diluted or undiluted either prior to or during the performance of the method. In some aspects, the dilution occurs prior to performing the methods. In other aspects, the dilution occurs during the performance of the method. In one aspect, the sample can be from about 1 to about 25 microliters, about 1 to about 24 microliters, about 1 to about 23 microliters, about 1 to about 22 microliters, about 1 to about 21 microliters, about 1 to about 20 microliters, about 1 to about 18 microliters, about 1 to about 17 microliters, about 1 to about 16 microliters, about 15 microliters or about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters or about 25 microliters. In some embodiments, the sample is from about 1 to about 150 microliters or less or from about 1 to about 25 microliters or less. Alternatively, in another aspect, the biological sample can be diluted about 1-fold, about 2-fold, about 3-fold, about 4-fold, about 5-fold, about 6-fold, about 10-fold, about 11-fold, about 12-fold, about 13-fold, about 15-fold, about 16-fold, about 17-fold, about 18-fold, about 19-fold, about 20-fold, about 21-fold, about 22-fold, about 23-fold, about 24-fold, about 25-fold, about 26-fold, about 27-fold, about 28-fold, about 29-fold, about 30-fold, about 31-fold, about 32-fold, about 33-fold, about 34-fold, about 35-fold, about 36-fold, about 37-fold, about 38-fold, about 39-fold, about 40-fold, about 41-fold, about 42-fold, about 43-fold, about 44-fold, about 45-fold, about 46-fold, about 47-fold, about 48-fold, about 49-fold, about 50-fold, about 100-fold, or greater prior to use (either before performing the method or during the performance of the method).

In some embodiments, the methods described herein further include treating and/or monitoring the subject identified as having anti-Rocahepevirus species 7 antibodies or antigen-binding fragments thereof. More specifically, the methods include administering an appropriate treatment and/or therapy to the subject if antibodies to Rocahepevirus species 7 or antigen-binding fragments thereof are detected in a sample. In some aspects, the subject can be treated with: (1) one or more anti-viral therapies (e.g., such as the administration of ribavirin, sofosbuvir, or combinations thereof); (2) one or more types of interferon (e.g., such as pegylated interferon-alpha); (3) hydration; (4) rest; or (5) any combination of (1)-(4).

The nature of methods described herein is not critical and can be performed using any assay known in the art such as, for example, described herein is not critical and the test can be any assay known in the art such as for example, immunoassays, protein immunoprecipitation, immunoelectrophoresis, chemical analysis, SDS-PAGE and Western blot analysis, or protein immunostaining, electrophoresis analysis, a protein assay, a competitive binding assay, a functional protein assay, or chromatography or spectrometry methods, such as high-performance liquid chromatography (HPLC) or liquid chromatography-mass spectrometry (LC/MS). Also, the assay can be employed in a clinical chemistry format such as would be known by one of ordinary skill in the art. It is known in the art that the values (e.g., reference levels, cutoffs, thresholds, specificities, sensitivities, concentrations of calibrators and/or controls etc.) used in an assay that employs specific sample type (e.g., such as an immunoassay that utilizes serum or a point-of-care device that employs whole blood) can be extrapolated to other assay formats using known techniques in the art, such as assay standardization. For example, one way in which assay standardization can be performed is by applying a factor to the calibrator employed in the assay to make the sample concentration read higher or lower to get a slope that aligns with the comparator method. Other methods of standardizing results obtained on one assay to another assay are well known and have been described in the literature (See, for example, David Wild, Immunoassay Handbook, 4^{th} edition, chapter 3.5, pages 315-322, the contents of which are herein incorporated by reference).

In some embodiments, the methods described herein can be implemented by a computer.

In yet further embodiments, the present disclosure relates to a computer-implemented method for evaluating or determining the presence of and/or the amount or concentration of anti-Rocahepevirus species 7 (genotype 1 or genotype 2) antibodies or antigen-binding fragments thereof in a sample obtained from a subject. The method comprises:
a. obtaining data on the presence of and/or amount or level of anti-Rocahepevirus species 7 (genotype 1 or genotype 2) antibodies or antigen-binding fragments thereof from one or more assays performed on at least one or more samples obtained from a subject;
b. analyzing the data to obtain to obtain an evaluation result; and
c. outputting the evaluation result, wherein the evaluation result is associated with whether the presence of anti-Rocahepevirus species 7 antibodies or antigen-binding fragments thereof were detected in one or more samples and/or the amount or concentration of anti-Rocahepevirus species 7 antibodies determined in the one or more samples. In some aspects, determining the presence of or amount or concentration of anti-Rocahepevirus species 7 antibodies or antigen-binding fragments thereof in one or more samples is performed according to the methods described herein, such as using an immunoassay. In still other aspects, determining the presence of or amount or concentration of anti-Rocahepevirus species 7 antibodies or antigen-binding fragments thereof in one or more samples is performed according to the methods described herein, such as using a clinical chemistry assay.

In still further aspects of the above computer-implemented method, the assay is a point-of-care assay. In still further aspects of the above computer-implemented method, the assay is a non-point-of-care assay. In still further aspects of the above computer-implemented method, the assay is a single molecule detection assay.

In yet further aspects of the above computer-implemented method, the sample is a blood sample. In yet further aspects, the blood sample is whole blood, serum, or plasma, either venous blood or capillary blood. In still yet further aspects, the blood sample is obtained from venous blood and is whole blood, serum, or plasma. In still yet other aspects, blood sample is obtained from capillary blood and is whole blood, serum, or plasma.

In still further aspects of the above computer-implemented method, the subject is a human subject.

### 3. Apparatus, Non-Transitory Machine-Readable Storage Medium, and Systems

In some aspects, disclosed herein are apparatus, machine-readable storage mediums and systems for use in implementing or performing the methods described in Section 2. More specifically, the apparatus, machine-readable storage mediums and systems described herein can be used to identify the presence of anti-Rocahepevirus species 7 antibodies or antigen-binding fragments thereof in one or more samples obtained from a subject, determine the amount or concentration of anti-Rocahepevirus species 7 antibodies or antigen-binding fragments thereof in the one or more samples, and communicate from an apparatus (e.g., such as a point-of-care, non-point-of-care, or a point-of-care or non-point-of-care apparatus), whether the level of anti-Rocahepevirus species 7 antibodies or antigen-binding fragments thereof, is greater than or less than one or more reference or other levels. The term “apparatus” is used interchangeably with “device” or “instrument”.

In another aspect, the present disclosure relates to an apparatus, device, or instrument. The apparatus, device, or instrument contains software to execute one or more tasks, including the performance of the methods described in Section 2. In some aspects, the apparatus, device, or instrument contains software to automatically determine the next appropriate step in a methods described herein. For example, the apparatus, device, or instrument may contain software that determines the amount or presence of an analyte of interest. The software may display this determination, such as on a graphical user interface.

In some aspects, the apparatus, device, or instruments stores software that instructs processor or processor circuitry to execute or instantiate a given task. In some aspects, the software stores machine-readable instructions that cause processor circuitry to execute or instantiate a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine-readable instructions and/or program(s) regardless of the particular format or state of the machine-readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C#, Perl, Python, JavaScript, HyperText Markup Language (HTML), Structured Query Language (SQL), Swift, etc.

The machine-readable instructions may be stored on a non-transitory computer and/or non-transitory machine-readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term “non-transitory computer readable medium” is defined to include any type of computer readable storage device and/or storage disk and to exclude propagating signals and to exclude transmission media.

In some further aspects, disclosed herein is a system for evaluating or determining (also referred to herein as an “evaluation system”) the presence of and/or the amount or concentration of anti-Rocahepevirus species 7 (genotype 1 or genotype 2) antibodies or antigen-binding fragments thereof in a sample obtained from a subject. The system comprises:
a. a data-obtaining module to obtain data on the presence of or amount or level of anti-Rocahepevirus species 7 (genotype 1 or genotype 2) antibodies or antigen-binding fragments thereof from one or more assays performed on at least one or more samples obtained from a subject;
b. an evaluation module to analyze the data to obtain an evaluation result is associated with whether the presence of anti-Rocahepevirus species 7 antibodies or antigen-binding fragments thereof were detected in one or more samples and/or the amount or concentration of anti-Rocahepevirus species 7 antibodies determined or detected in the one or more samples; and
c. a data-outputting module to output the evaluation result,
wherein the first evaluation result is associated with determining whether the presence of anti-Rocahepevirus species 7 antibodies or antigen-binding fragments thereof were detected in the sample and/or the amount or concentration of anti-Rocahepevirus species 7 antibodies detected in the sample obtained from the subject.

In other aspects of the above evaluation system, the presence of and/or measurement of the amount or concentration of anti-Rocahepevirus species 7 antibodies or antigen-binding fragments thereof is detected by immunoassay. In still further aspects of the above evaluation system, detecting the presence of or measuring the amount or concentration of anti-Rocahepevirus species antibodies or antigen-binding fragments thereof is detected by a clinical chemistry assay.

In still further aspects of the above evaluation system, the assay is a point-of-care assay, a non-point-of-care assay, or a single molecule detection assay.

In still further aspects of the above evaluation system, the sample is a blood sample. In yet further aspects, the blood sample is whole blood, serum, or plasma, either venous blood or capillary blood. In still yet further aspects, the blood sample is obtained from venous blood and is whole blood, serum, or plasma. In still yet other aspects, blood sample is obtained from capillary blood and is whole blood, serum, or plasma.

In still further aspects of the above evaluation system, the subject is a human subject.

In further aspects, disclosed herein is a computer device that comprises a storage device having machine-readable instructions stored thereon, and a processor. The processor executes the machine-readable instructions to perform the steps of the above computer-implemented method or the steps performed by the above evaluation system.

In further aspects, disclosed herein is an apparatus, device, or instrument that comprises a storage device having machine-readable instructions stored thereon, and a processor. The processor instantiates or executes the machine-readable instructions to:
(A) identify the presence of and/or amount or concentration of anti-Rocahepevirus species 7 antibodies or antigen-binding fragments thereof in at least one or more biological samples obtained from a subject using at least one assay;
(B) determine the presence of and/or amount or concentration of anti-Rocahepevirus species 7 antibodies or antigen-binding fragments thereof in one or more samples, and
(C) communicate from the apparatus, device, or instrument (e.g., such as displaying on the apparatus, device, or instrument), (i) the presence or absence of one or more of anti-Rocahepevirus species 7 antibodies or antigen-binding fragments thereof in the sample; (ii) the amount or concentration of one or more of anti-Rocahepevirus species 7 antibodies or antigen-binding fragments thereof in the sample; (iii) whether the level of one or more of anti-Rocahepevirus species 7 antibodies or antigen-binding fragments thereof is greater than or less than a reference level; or (iv) any combination of (i)-(iii).

In further aspects, disclosed herein is a non-transitory machine-readable storage medium having machine-readable instructions stored thereon, wherein the machine-readable instructions are configured to be executed by a processor to perform the steps of the above-described computer-implemented method or the steps performed by the above one or more systems, such as the evaluation system.

In yet further aspects, disclosed herein is a non-transitory machine-readable storage medium. The non-transitory machine-readable storage medium can comprise instructions to cause one or processors or processor circuitry to at least:
(A) identify the presence of and/or amount or concentration of anti-Rocahepevirus species 7 antibodies or antigen-binding fragments thereof in at least one or more samples obtained from a subject using at least one assay;
(B) determine the presence of and/or amount or concentration of anti-Rocahepevirus species 7 antibodies or antigen-binding fragments thereof in one or more samples, and
(C) communicate from the apparatus, device, or instrument (e.g., such as displaying on the apparatus, device, or instrument), (i) the presence or absence of one or more of anti-Rocahepevirus species 7 antibodies or antigen-binding fragments thereof in the sample obtained from the subject; (ii) the amount or concentration of one or more of anti-Rocahepevirus species 7 antibodies or antigen-binding fragments thereof in the sample obtained from the subject; (iii) whether the level of one or more of anti-Rocahepevirus species 7 antibodies or antigen-binding fragments thereof in the sample obtained from the subject is greater than or less than a reference level; or (iv) any combination of (i)-(iii).

In still yet further aspects disclosed herein are systems. In some aspects, the system comprises an apparatus, device, or instrument and certain instructions. With respect to the apparatus, the apparatus, device, or instrument comprises a storage device having machine readable instructions stored thereon and a processor or processor circuitry which instantiates or executes the machine-readable instructions to:
(A) identify the presence of and/or amount or concentration of anti-Rocahepevirus species 7 antibodies in at least one or more samples obtained from a subject using at least one assay;
(B) determine the presence of and/or amount or concentration of anti-Rocahepevirus species 7 antibodies one or more samples obtained from the subject, and
(C) communicate from the apparatus, device, or instrument (e.g., such as displaying on the apparatus, device, or instrument), (i) the presence or absence of one or more of anti-Rocahepevirus species 7 antibodies in the sample obtained from the subject; (ii) the amount or concentration of one or more of anti-Rocahepevirus species 7 antibodies or antigen-binding fragments thereof in the sample obtained from the subject; (iii) whether the level of one or more of anti-Rocahepevirus species 7 antibodies or antigen-binding fragments thereof is greater than or less than a reference level; or (iv) any combination of (i)-(iii).

In addition to the apparatus, device, or instrument, the system also comprises instructions. The instructions included in the system are for use in determining:
(A) whether the sample obtained from the subject contains one or more anti-Rocahepevirus species 7 antibodies or antigen-binding fragments thereof in at least one or more samples obtained from a subject using at least one assay;
(B) if present, the amount or concentration of one or more anti-Rocahepevirus species 7 antibodies or antigen-binding fragments thereof in the sample obtained from the subject;
(C) whether the amount, concentration or level of one or more of anti-Rocahepevirus species 7 antibodies or antigen-binding fragments thereof is greater than or less than a reference level;
(D) any combination of (A)-(C).

The instructions that are included in the system can be provided as written instructions, such as in a product insert, product manual, excel document, etc., on a mobile device (e.g., a smart phone), on a website, in an e-mail, or any combination thereof.

### 4. Kit

Provided herein is a kit, which may be used for assaying or assessing a sample for one or more anti-Rocahepevirus species 7 (genotype 1 or genotype 2) antibodies. The kit comprises at least one component for assaying the test sample for Rocahepevirus and instructions for assaying the sample anti-Rocahepevirus species 7 antibodies or antigen-binding fragments thereof. For example, the kit can comprise instructions for assaying the sample for anti-Rocahepevirus species 7 antibodies or antigen-binding fragments thereof by immunoassay, e.g., chemiluminescent microparticle immunoassay. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The at least one component may include at least one composition comprising one or more isolated recombinant antigens or isolated polypeptides (e.g., such as any of SEQ ID NO:1 or any variant or fragment thereof, SEQ ID NO:2 or any variant or fragment thereof, SEQ ID NO: 3 or any variant or fragment thereof, and/or SEQ ID NO:4 or any variant or fragment thereof) that specifically bind to anti-Rocahepevirus species antibodies or antigen-binding fragments thereof. The one or more isolated recombinant antigens or isolated polypeptides may be one or more of the four types of capture specific binding partners and/or the at least one type of detection specific binding partner.

Alternatively or additionally, the kit can comprise a calibrator or control and/or at least one container (e.g., tube, microtiter plates or strips) for conducting the methods or assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

The kit may further comprise reference standards for quantifying the anti-Rocahepevirus species antibodies identified in a sample.

The kit can also contain capture specific binding partners, such as, a first capture specific binding partner comprising or consisting of SEQ ID NO:1 or a fragment thereof, a second capture specific binding partner comprising or consisting of SEQ ID NO:2 or a fragment thereof, a third specific binding partner comprising or consisting of SEQ ID NO:3 or a fragment thereof, and/or a fourth specific binding partner comprising or consisting of SEQ ID NO:4 or a fragment thereof.

The kit may further comprise at least one detection specific binding partner. The at least one detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) and an anti-species IgM (e.g., anti-human-IgM IgG) antibody. Optionally, the at least one detection specific binding partner can incorporate a detectable label, such as a fluorophore, radioactive moiety, enzyme, biotin/avidin label, chromophore, chemiluminescent label, or the like, or the kit can include reagents for labeling the at least one detection specific binding partner. The capture specific binding partners, detection specific binding partners, calibrators, and/or controls can be provided in separate containers or pre-dispensed into an appropriate assay format, for example, into microtiter plates,

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase or solid support, such as a magnetic particle, bead, test tube, microtiter plate, cuvette, membrane, scaffolding molecule, film, filter paper, disc, or chip.

By way of example, and not of limitation, examples of the present disclosures shall now be given.

The present disclosure has multiple aspects, illustrated by the following non-limiting examples.

### Example 1

FIG. 3 provides an example of a 2-step indirect IgG assay that can be made using any of SEQ ID NOS: 1-8 to detect Rocahepevirus species 7, genotype 1 or genotype 2 in a sample obtained from subject, such as a human, suspected of having or having Rocahepevirus. As shown in FIG. 3, each of SEQ ID NOS: 1-4, SEQ ID NO:5-8, or a combination of SEQ ID NOS: 1-4 and 5-8, can be immobilized on a solid support, such as a bead or microparticle. In some aspects, a solid support can be immobilized with each of SEQ ID NOS: 1-4 as a first capture specific binding partner, a second capture specific binding partner, a third capture specific binding partner, or a fourth capture specific binding partner, respectively. Alternatively, in some aspects, a solid support can be immobilized with each of SEQ ID NOS: 5-8 as a first capture specific binding partner, a second capture specific binding partner, a third capture specific binding partner, or a fourth capture specific binding partner, respectively. Alternatively, in some aspects, a solid support can be immobilized with a first capture specific binding partner having an amino acid sequence of SEQ ID NO:1, a second capture specific binding partner having an amino acid sequence of SEQ ID NO:6, a third capture specific binding partner having an amino acid sequence of SEQ ID NO:7, and a further capture specific binding partner having an amino acid sequence of SEQ ID NO:4. After the at least four capture specific binding partners are immobilized on the solid support (“the capture reagent”), the capture reagent can be placed in contact with the sample. Optionally, after a few minutes of incubation, at least one detection specific binding partner can be added. At least one detection-specific binding partner is labeled with at least one detectable label (“the detection reagent”). The at least one detection reagent can be an anti-human IgG antibody. Optionally, after a few minutes of incubation, the resulting capture reagent-anti-Rocahepevirus species 7 antibody-detection reagent complexes can be detected using routine techniques known in the art. Using such an assay the presence of or amount or concentration of Rocahepevirus species 7 can be determined in a sample obtained from a subject.

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the present disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the present disclosure, may be made without departing from the spirit and scope thereof.

For reasons of completeness, various aspects of the disclosure are set out in the following numbered clauses:

Clause 1. A method for detecting a presence of or determining an amount of a Rocahepevirus species 7 antibody or an antigen-binding fragment thereof in a subject, the method comprising the steps of:
a) contacting at least one biological sample from the subject, either simultaneously or sequentially, in any order, with
at least four types of capture specific binding partners, wherein each of the four capture specific binding partners specifically binds to a Rocahepevirus species 7 antibody or an antigen-binding fragment thereof in the sample; and further wherein the four types of capture specific binding partners comprises: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof, and
at least one type of detection specific binding partner comprising a detectable label,
thereby producing one or more types of first complexes comprising the four capture specific binding partners-anti-Rocahepevirus species 7 antibody-detection specific binding partner; and
b) assessing a signal from the one or more types of first complexes, wherein the amount of detectable signal from the detectable label indicates the presence or amount of at least one type of anti-Rocahepevirus species 7 antibody or an antigen-binding fragment thereof in the sample.

Clause 2. The method of clause 1, wherein the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

Clause 3. The method of clause 1 or clause 2, wherein the biological sample is whole blood, plasma, or serum.

Clause 4. The method of any of clauses 1-3, wherein the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) and an anti-species IgM (e.g., anti-human-IgM IgG) antibody.

Clause 5. The method of any of clauses 1-4, wherein the each of the four capture specific binding partner are immobilized on a solid support.

Clause 6. The method of any of clauses 1-5, wherein the method is performed in from about 5 to about 20 minutes, less than about 20 minutes, and optionally is performed in less than about 5 minutes, less than about 10 minutes or less than about 15 minutes.

Clause 7. The method of any of clauses 1-6, wherein the method further comprises use with at least one calibrator reagent, at least one control reagent, or at least one calibrator reagent and at least one control reagent.

Clause 8. The method of any of clauses 1-7, wherein the method is an immunoassay or a clinical chemistry assay.

Clause 9. The method of any of clauses 1-8, wherein the method is performed using single molecule detection, lateral flow assay, or a point-of-care assay.

Clause 10. A kit for performing the method of clause 1, wherein the kit comprises:
a. at least four types of capture specific binding partners, wherein each of the four capture specific binding partners specifically binds to a Rocahepevirus species 7 antibody or an antigen-binding fragment thereof in a sample; and further wherein the four types of capture specific binding partners comprise: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof; and
b. at least one type of detection specific binding partner comprising at least one detectable label.

Clause 11. The kit of clause 10, wherein the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

Clause 12. The kit of clause 10 or clause 11, wherein the kit further comprises, or is configured to be used with, at least one calibrator reagent, at least one control reagent, or at least one calibrator reagent and at least one control reagent.

Clause 13. The kit of any of clauses 10-12, wherein the kit further comprises at least one solid support.

Clause 14. The kit of any of clauses 10-13, wherein the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) antibody and an anti-species IgM (e.g., anti-human-IgM IgG) antibody.

Clause 15. The kit of any of clauses 10-14, wherein the kit is adapted for use with an automated or semi-automated system.

1. A method for detecting a presence of or determining an amount of a Rocahepevirus species 7 antibody or an antigen-binding fragment thereof in a subject, the method comprising the steps of:
a) contacting at least one biological sample from the subject, either simultaneously or sequentially, in any order, with
at least four types of capture specific binding partners, wherein each of the four capture specific binding partners specifically binds to a Rocahepevirus species 7 antibody or an antigen-binding fragment thereof in the sample; and further wherein the four types of capture specific binding partners comprises: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof, and
at least one type of detection specific binding partner comprising a detectable label,
thereby producing one or more types of first complexes comprising the four capture specific binding partners-anti-Rocahepevirus species 7 antibody-detection specific binding partner; and
b) assessing a signal from the one or more types of first complexes, wherein the amount of detectable signal from the detectable label indicates the presence or amount of at least one type of anti-Rocahepevirus species 7 antibody or an antigen-binding fragment thereof in the sample.

2. The method of claim 1, wherein the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

3. The method of claim 1, wherein the biological sample is whole blood, plasma, or serum.

4. The method of claim 1, wherein the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) and an anti-species IgM (e.g., anti-human-IgM IgG) antibody.

5. The method of claim 1, wherein the each of the four capture specific binding partner are immobilized on a solid support.

6. The method of claim 1, wherein the method is performed in from about 5 to about 20 minutes, less than about 20 minutes, and optionally is performed in less than about 5 minutes, less than about 10 minutes or less than about 15 minutes.

7. The method of claim 1, wherein the method further comprises use with at least one calibrator reagent, at least one control reagent, or at least one calibrator reagent and at least one control reagent.

8. The method of claim 1, wherein the method is an immunoassay or a clinical chemistry assay.

9. The method of claim 1, wherein the method is performed using single molecule detection, lateral flow assay, or a point-of-care assay.

10. A kit for performing the method of claim 1, wherein the kit comprises:
a. at least four types of capture specific binding partners, wherein each of the four capture specific binding partners specifically binds to a Rocahepevirus species 7 antibody or an antigen-binding fragment thereof in a sample; and further wherein the four types of capture specific binding partners comprise: (i) a first capture specific binding partner comprising an isolated polypeptide having an amino acid sequence of SEQ ID NO:1 or a fragment thereof; (ii) a second capture specific binding partner comprising an isolated polypeptide having an amino acid sequence of SEQ ID NO:2 or a fragment thereof; (iii) a third capture specific binding partner comprising an isolated polypeptide having an amino acid sequence of SEQ ID NO:3 or a fragment thereof; and (iv) a fourth capture specific binding partner comprising an isolated polypeptide having an amino acid sequence of SEQ ID NO: 4 or a fragment thereof; and
b. at least one type of detection specific binding partner comprising at least one detectable label.

11. The kit of claim 10, wherein the fragment of the first capture specific binding partner comprises an amino acid sequence of SEQ ID NO:5, the fragment of the second capture specific binding partner comprises an amino acid sequence of SEQ ID NO:6, the fragment of the third specific binding partner has an amino acid sequence of SEQ ID NO:7, and the fragment of the fourth specific binding partner has an amino acid sequence of SEQ ID NO:8.

12. The kit of claim 10, wherein the kit further comprises, or is configured to be used with, at least one calibrator reagent, at least one control reagent, or at least one calibrator reagent and at least one control reagent.

13. The kit of claim 10, wherein the kit further comprises at least one solid support.

14. The kit of claim 10, wherein the at least one type of detection specific binding partner is an anti-species IgG (e.g., anti-human-IgG IgG) antibody, an anti-species IgM (e.g., anti-human-IgM IgG) antibody, or an anti-species IgG (e.g., anti-human-IgG IgG) antibody and an anti-species IgM (e.g., anti-human-IgM IgG) antibody.

15. The kit of claim 10, wherein the kit is adapted for use with an automated or semi-automated system.


SEQUENCE LISTING (8 sequences)

SEQ ID NO: 1 - protein, 644 aa, Rocahepevirus sp.
  source 1..644: note = species 7
MRVTFVLVLLLFVPLLFAAPMYPGGTRGAGGPRRGTRADATVPHPAPPPIQRTNWLSPTNPFAAELSTMPPLGSAWVDRRRGRPRNRVTIVPDAGQSPMTAQAPAPNTAPVPDVDSRGSILRRQYNLSTSPLTLTVPQSSNSILYAASLNPLLPLQDGSNTHIMATEASNYAQYRVQAATLRFRPLVPNSVGGYAVSMSFWPQSTTVPTSVDMNSITSTDVRIVAQPGLAAELVIPRERLHYRNQGWRSVETNSVPQEESTSGMLMVCIHGAPINSFTNSAYTGALGLLDIALQVEYRNLTPGNTNTRVNRFRTTAPHKIKRMPDGTAQVATASAVRFMQDVHLTGGNGVGDIGKGIVQVLFNIADTLLGGLPTDLVSNAGGQLFYGRPQVSENGEPSVKLYTSVEAAQLDQGVTIPHDIDLGLSAVTLQDFDNQHLQDRPTPSPAPARPITNWRSGDVVWVTLPSAEYAQSQSAMASHPAYWSEEATIINVATGQRAAVSSIKWDQVTLNGKALHKETHSGLVYYQLPLMGKINFWQQGTTKAGYTYNYNTTDSDSLWVWWDGSSKAYLYISTYTTMLGAGPVNITGLGAIGPSPTDQASVAVPMHNLQDGCPACALAGLSTCLVADAMARVQALSAAAADSV

SEQ ID NO: 2 - protein, 644 aa, Rocahepevirus sp.
  source 1..644: note = species 7
MRVAFVLVLLLFVPLLLAAPMYPGGTRGVGGPRRGTRSDGAVPHPAQPPPQRTNWLSPTNPFAAELSTMPPLGTAWVDRRRGKPRNRVAVVPDAGQSPMTAQAPAPNTAPVPDVDSRGSILRRQYNLSTSPLTLTVPQSSNSILYAASLNPLLPLQDGSNTHIMATEASNYAQYRVQACTLRFRPLVPNSVGGYAVSMSFWPQSTTVPTSVDMNSITSTDVRIVAQPGLAAELVVPRERLHYRNQGWRSVETNSVPQEESTSGMLMVCIHGAPINSFTNSAYTGALGLLDIALQVEYRNLTPGNTNTRVNRFRTTAPHKIKRLPDGTAQVATASAVRFMQDVHLTGGNGVGDIGKGIVQVLFNIADTLLGGLPTDLVSNAGGQLFYGRPQVSENGEPSVKLYTSVEAAQLDQGVTIPHDIDLGLSAVTLQDFDNQHLQDRPTPSPAPARPITNWRSGDVVWVTLPSAEYAQSQSAMGSHPAYWSEEATIINVATGQRALVSSIKWDQVTLNGKALHKETHSGLVYYQLPLMGKISFWQQGTTKAGYTYNYNTTDSDSLWVWWDGTSKAYLYVSTYTTMLGAGPVNITGLGAIGPSPTEQASVAAPMHNLQDGCPACALAGLSSCLVADAMARVQALSAAAADSV

SEQ ID NO: 3 - protein, 644 aa, Rocahepevirus sp.
  source 1..644: note = species 7
MSVVVVLVLLLFVPLLPAAPMYPGGPRGAGGPRRGTRADAALPHPTLPPTQHVNWLSPTNPFAAELSRVPPLGSAWVDRRRGGSRRRAAPLSDAGQSPMTAQAPAPNTAPVPDVDSRGAILRRQYNLSTSPLTLTVPQSSNSILYAASLNPLLPLQDGSNTHIMATEASNYAQYRVQAATLRFRPLVPNSVGGYAVSMSFWPQSTTVPTSVDMNSITSTDVRIVAQPGLAAELVIPKERLHYRNQGWRSVETNSVPQEESTSGMLMVCIHGAPINSFTNTAYTGALGLLDIALQVEYRNLTPGNTNTRVNRFRTTAPHKIKRLPDGTAQVSTASAVRFMQDVHLSGNNGVGDIGKGIVQVLFNIADTLLGGLPTDLVSNAGGQLFYGRPQVSENGEPSVKLYTSVEAAQLDHGVTIPHDIDLGVSAVTLQDFDNQHLQDRPTPSPAPARPITNWRSGDVVWVTLPSAEYIQSQSAMGSHPAYWSEEATIINVATGQRALVSSIKWDQVTLNGKALHKETHSGLVYYQLPLMGKINFWQQGTTRAGYTYNYNTTDSDSIWVWWDGGSKAYLYISTYTTMLGAGPVNITGLGAVGPNPTDQASAVSPMHNLQDGCPACALAGLSTCLVADAMARVQALSTAAADSV

SEQ ID NO: 4 - protein, 644 aa, Rocahepevirus sp.
  source 1..644: note = species 7
MSVVVVLVLLLFVPLLSAAPMYPGGPRGAGGPRRGTRADAALSHPAPPPTQHTNWLSPTNPFAAELSRVPPLGSAWVDRRRGGPRRRSAPVSDAGQSPMTAQAPAPNTAPVPDVDSRGSILRRQYNLSTSPLTLTVPQSSNSILYAASLNPLLPLQDGSNTHIMATEASNYAQYRVQAATLRFRPLVPNSVGGYAVSMSFWPQSTTVPTSVDMNSITSTDVRIVAQPGLAAELVIPKERLHYRNQGWRSVETNSVPQEESTSGMLMVCIHGAPINSFTNTPYTGALGLLDIALQVEYRNLTPGNTNTRVNRFRTTAPHKIKRLPDGTAQVSTASAVRFMQDVHLAGNNGVGDIGKGIVQVLFNIADTLLGGLPTDLVSNAGGQLFYGRPQVSENGEPSVKLYTSVEAAQLDHGVTIPHDIDLGVSAITLQDFDNQHLQDRPTPSPAPARPITNWRSGDVVWVTLPSAEYAQSQSAMGSHPAYWSEEATIINVATGQRAAVSSIKWDQVTLNGKALHKETHSGLVYYQLPLMGKINFWQQGTTKAGYTYNYNTTDSDSLWVWWDGGSKAYLYISTYTTMLGAGPVNITGLGAVGPNPVDQASAVVPMHNLQDGCPACALAGLSSCLVADAMARVQALSAAAADSV

SEQ ID NO: 5 - protein, 165 aa, Rocahepevirus sp.
  source 1..165: note = species 7
DFDNQHLQDRPTPSPAPARPITNWRSGDVVWVTLPSAEYAQSQSAMASHPAYWSEEATIINVATGQRAAVSSIKWDQVTLNGKALHKETHSGLVYYQLPLMGKINFWQQGTTKAGYTYNYNTTDSDSLWVWWDGSSKAYLYISTYTTMLGAGPVNITGLGAIGPS

SEQ ID NO: 6 - protein, 165 aa, Rocahepevirus sp.
  source 1..165: note = species 7
DFDNQHLQDRPTPSPAPARPITNWRSGDVVWVTLPSAEYAQSQSAMGSHPAYWSEEATIINVATGQRALVSSIKWDQVTLNGKALHKETHSGLVYYQLPLMGKISFWQQGTTKAGYTYNYNTTDSDSLWVWWDGTSKAYLYVSTYTTMLGAGPVNITGLGAIGPS

SEQ ID NO: 7 - protein, 165 aa, Rocahepevirus sp.
  source 1..165: note = species 7
DFDNQHLQDRPTPSPAPARPITNWRSGDVVWVTLPSAEYIQSQSAMGSHPAYWSEEATIINVATGQRALVSSIKWDQVTLNGKALHKETHSGLVYYQLPLMGKINFWQQGTTRAGYTYNYNTTDSDSIWVWWDGGSKAYLYISTYTTMLGAGPVNITGLGAVGPN

SEQ ID NO: 8 - protein, 165 aa, Rocahepevirus sp.
  source 1..165: note = species 7
DFDNQHLQDRPTPSPAPARPITNWRSGDVVWVTLPSAEYAQSQSAMGSHPAYWSEEATIINVATGQRAAVSSIKWDQVTLNGKALHKETHSGLVYYQLPLMGKINFWQQGTTKAGYTYNYNTTDSDSLWVWWDGGSKAYLYISTYTTMLGAGPVNITGLGAVGPN